(19) United States
(12) Patent Application Publication
(10) Pub. No.: US 2023/0103685 A1
(43) Pub. Date: Apr. 6, 2023

(54) IODINE-CONTAINING ACID CLEAVABLE COMPOUNDS, POLYMERS DERIVED THEREFROM, AND PHOTORESIST COMPOSITIONS

(71) Applicant: ROHM AND HAAS ELECTRONIC MATERIALS LLC, Marlborough, MA (US)

(72) Inventors: Emad Aqad, Northborough, MA (US); Jong Keun Park, Shrewsbury, MA (US); Bhooshan C. Popere, Sturbridge, MA (US); Li Cui, Westborough, MA (US); Yinjie Cen, Shrewsbury, MA (US); Choong-Bong Lee, Westborough, MA (US)

(21) Appl. No.: 17/490,923

(22) Filed: Sep. 30, 2021

(51) Int. Cl. G03F 7/039; G03F 7/004; G03F 7/20; G03F 7/16; G03F 7/32

(52) U.S. Cl. CPC G03F 7/0392; G03F 7/0045; G03F 7/2006; G03F 7/162; G03F 7/327

(57) ABSTRACT

A compound comprising an aromatic group or a heteroaromatic group, wherein the aromatic group or the heteroaromatic group comprises a first substituent group comprising an ethylenically unsaturated double bond, a second substituent group that is an iodine atom, and a third substituent group comprising an acid-labile group, wherein the first substituent group, the second substituent group, and the third substituent group are each bonded to a different carbon atom of the aromatic group or the heteroaromatic group.

### FIELD

The present invention relates to iodine-containing acid-cleavable compounds, polymers derived from these compounds, photoresist compositions including such polymers, and pattern formation methods using such photoresist compositions. The invention finds particular applicability in lithographic applications in the semiconductor manufacturing industry.

BACKGROUND

Photoresist compositions are photosensitive materials used to transfer a pattern to one or more underlying layers, such as a metal, semiconductor, or dielectric layer disposed on a substrate. Positive-tone chemically amplified photoresist compositions are conventionally used for high-resolution processing. Such resist compositions typically include a polymer having acid-labile groups and a photoacid generator (PAG). A layer of the photoresist composition is pattern-wise exposed to activating radiation and the PAG generates an acid in the exposed regions. During post-exposure baking, the acid causes cleavage of the polymer's acid-labile groups. This creates a difference in solubility characteristics between exposed and unexposed regions of the photoresist layer in a developer solution. In a positive tone development (PTD) process, exposed regions of the photoresist layer become soluble in a developer, typically an aqueous base developer, and are removed from the substrate surface. Unexposed regions, which are insoluble in the developer, remain after development to form a positive relief image. The resulting relief image permits selective processing of the substrate.

To increase the integration density of semiconductor devices and allow for the formation of structures having dimensions in the nanometer (nm) range, photoresists and photolithography processing tools having high-resolution capabilities have been and continue to be developed. One approach to achieving nm-scale feature sizes in semiconductor devices is the use of activating radiation having a short wavelength, for example, 193 nm or less, for exposure of the photoresist layer. To further improve lithographic performance, immersion lithography tools have been developed to effectively increase the numerical aperture (NA) of the lens of the imaging device. This is accomplished by use of a relatively high refractive index fluid, typically water, between the last surface of the imaging device and the upper surface of the semiconductor wafer.

Deep-ultraviolet argon fluoride (ArF) excimer-laser immersion tools are currently pushing the boundaries of lithographic processing to the 16 nm and 14 nm device nodes with the use of multiple (double, triple, or higher order) patterning techniques. The use of multiple patterning, however, can be costly in terms of increased material usage and number of process steps required as compared with single step, directly-imaged patterns. The need for photoresist compositions for next-generation (e.g., Extreme Ultraviolet, EUV) lithography, which use activating radiation having an extremely short wavelength of 13.5 nm, has thus become of increased importance for advanced device nodes. At the extreme feature sizes associated with these nodes, the performance requirements of photoresist compositions have become increasingly more stringent. Desired performance properties include, for example, high sensitivity to activating radiation, low unexposed film thickness loss (UFTL), good contrast, high-resolving capability, and good line-width roughness (LWR).

One way to increase EUV photoresist sensitivity is by increasing the absorption cross section at 13.5 nm. The material absorption at 13.5 nm is an atomic property and can be theoretically calculated using known atomic absorptions. Typical atoms that make up of resist materials, such as carbon, oxygen, hydrogen, and nitrogen possess very weak EUV absorption. Fluorine atom has slightly higher absorption and has been used in the search for high EUV absorbing photoresist. Iodine has a remarkably high absorption cross section at EUV radiation. JP 2015-161823; US 10,095,109 B1; and U.S. Pat. No. 10,495,968 B2 describe iodine-containing monomers and corresponding polymers useful for lithographic processing. In addition, JP 2018-95851 A describes iodine-containing monomers that have carboxylic acid groups. However, attaching the highly alkali soluble carboxylic acid group can lead to unexposed film thickness loss and therefore to poor resolution.

Accordingly, there remains a continued need in the art for acid-labile compounds to provided EUV photoresist polymers having good absorption at 13.5 nm, reduced unexposed film thickness loss (UFTL), improved LWR, or a combination thereof.

### SUMMARY

Provided is a compound comprising an aromatic group or a heteroaromatic group, wherein the aromatic group or the heteroaromatic group comprises a first substituent group comprising an ethylenically unsaturated double bond, a second substituent group that is an iodine atom, and a third substituent group comprising an acid-labile group, wherein the first substituent group, the second substituent group, and the third substituent group are each bonded to a different carbon atom of the aromatic group or the heteroaromatic group.

Also provided is a polymer comprising a first repeating unit derived from the inventive compound. Another aspect provides a photoresist composition comprising the inventive polymer, a photoacid generator (PAG), and a solvent.

Yet another aspect provides a method for forming a pattern comprising applying a layer of the inventive photoresist composition on a substrate to provide a photoresist composition layer; pattern-wise exposing the photoresist composition layer to activating radiation to provide an exposed photoresist composition layer; and developing the exposed photoresist composition layer to provide a photoresist pattern.

### DETAILED DESCRIPTION

Reference will now be made in detail to exemplary embodiments, examples of which are illustrated in the present description. In this regard, the present exemplary embodiments may have different forms and should not be construed as being limited to the descriptions set forth herein.

Accordingly, the exemplary embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

As used herein, the terms “a,” “an,” and “the” do not denote a limitation of quantity and are to be construed to cover both the singular and the plural, unless otherwise indicated herein or clearly contradicted by context. “Or” means “and/or” unless clearly indicated otherwise. The modifier “about” used in connection with a quantity is inclusive of the stated value and has the meaning dictated by the context (e.g., includes the degree of error associated with measurement of the particular quantity). All ranges disclosed herein are inclusive of the endpoints, and the endpoints are independently combinable with each other. The suffix “(s)” is intended to include both the singular and the plural of the term that it modifies, thereby including at least one of that term. “Optional” or “optionally” means that the subsequently described event or circumstance can or cannot occur, and that the description includes instances where the event occurs and instances where it does not. The terms “first,” “second,” and the like, herein do not denote an order, quantity, or importance, but rather are used to distinguish one element from another. When an element is referred to as being “on” another element, it may be directly in contact with the other element or intervening elements may be present therebetween. In contrast, when an element is referred to as being “directly on” another element, there are no intervening elements present. It is to be understood that the described components, elements, limitations, and/or features of aspects may be combined in any suitable manner in the various aspects.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “actinic rays” or “radiation” means, for example, a bright line spectrum of a mercury lamp, far ultraviolet rays represented by an excimer laser, extreme ultraviolet rays (EUV light), X-rays, particle rays such as electron beams and ion beams, or the like. In addition, in the present invention, “light” means actinic rays or radiation. The krypton fluoride laser (KrF laser) is a particular type of excimer laser, which is sometimes referred to as an exciplex laser. “Excimer” is short for “excited dimer,” while “exciplex” is short for “excited complex.” An excimer laser uses a mixture of a noble gas (argon, krypton, or xenon) and a halogen gas (fluorine or chlorine), which under suitable conditions of electrical stimulation and high pressure, emits coherent stimulated radiation (laser light) in the ultraviolet range. Furthermore, “exposure” in the present specification includes, unless otherwise specified, not only exposure by a mercury lamp, far ultraviolet rays represented by an excimer laser, X-rays, extreme ultraviolet rays (EUV light), or the like, but also writing by particle rays such as electron beams and ion beams.

As used herein, the term “hydrocarbon” refers to an organic compound or group having at least one carbon atom and at least one hydrogen atom; “alkyl” refers to a straight or branched chain saturated hydrocarbon group having the specified number of carbon atoms and having a valence of one; “alkylene” refers to an alkyl group having a valence of two; “hydroxyalkyl” refers to an alkyl group substituted with at least one hydroxyl group (—OH); “alkoxy” refers to “alkyl-O—”; “carboxyl” and “carboxylic acid group” refer to a group having the formula “—C(═O)—OH”; “cycloalkyl” refers to a monovalent group having one or more saturated rings in which all ring members are carbon; “cycloalkylene” refers to a cycloalkyl group having a valence of two; “alkenyl” refers to a straight or branched chain, monovalent hydrocarbon group having at least one carbon-carbon double bond; “alkenoxy” refers to “alkenyl-O—”; “alkenylene” refers to an alkenyl group having a valence of two; “cycloalkenyl” refers to a non-aromatic cyclic divalent hydrocarbon group having at least three carbon atoms, with at least one carbon-carbon double bond; “alkynyl” refers to a monovalent hydrocarbon group having at least one carbon-carbon triple bond; the term “aromatic group” refers to a monocyclic or polycyclic aromatic ring system that satisfies Huckel's Rule (4n+2 π electrons) and includes carbon atoms in the ring; the term “heteroaromatic group” refers to an aromatic group that includes one or more heteroatoms (e.g., 1-4 heteroatoms) selected from N, O, and S instead of a carbon atom in the ring; “aryl” refers to a monovalent monocyclic or polycyclic aromatic ring system where every ring member is carbon, and may include a group with an aromatic ring fused to at least one cycloalkyl or heterocycloalkyl ring; “arylene” refers to an aryl group having a valence of two; “alkylaryl” refers to an aryl group that has been substituted with an alkyl group; “arylalkyl” refers to an alkyl group that has been substituted with an aryl group; “aryloxy” refers to “aryl-O—”; and “arylthio” refers to “aryl—S—”.

The prefix “hetero” means that the compound or group includes at least one member that is a heteroatom (e.g., 1, 2, 3, or 4 or more heteroatom(s)) instead of a carbon atom, wherein the heteroatom(s) is each independently N, O, S, Si, or P; “heteroatom-containing group” refers to a substituent group that includes at least one heteroatom; “heteroalkyl” refers to an alkyl group having at least one heteroatom instead of carbon.

The term “heterocycloalkyl” refers to a cycloalkyl group having at least one heteroatom that is chosen independently from N, O, or S as a ring member instead of carbon; “heterocycloalkylene” refers to a heterocycloalkyl group having a valence of two. Exemplary 3-membered heterocycloalkyl groups containing 1 heteroatom include azirdinyl, oxiranyl, and thiiranyl. Exemplary 4-membered heterocycloalkyl groups containing 1 heteroatom include azetidinyl, oxetanyl, and thietanyl. Exemplary 5-membered heterocycloalkyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocycloalkyl groups containing 2 heteroatoms include dioxolanyl, oxathiolanyl, and dithiolanyl. Exemplary 5-membered heterocycloalkyl groups containing 3 heteroatoms include triazolinyl, oxadiazolinyl, and thiadiazolinyl. Exemplary 6-membered heterocycloalkyl groups containing 1 heteroatom include piperidinyl, tetrahydropyranyl, dihydropyridinyl, and thianyl. Exemplary 6-membered heterocycloalkyl groups containing 2 heteroatoms include piperazinyl, morpholinyl, dithianyl, and dioxanyl. Exemplary 6-membered heterocycloalkyl groups containing 2 heteroatoms include triazinanyl. Exemplary 7-membered heterocycloalkyl groups containing 1 heteroatom include azepanyl, oxepanyl, and thiepanyl. Exemplary 8-membered heterocycloalkyl groups containing 1 heteroatom include azocanyl, oxecanyl and thiocanyl. Exemplary bicyclic heterocycloalkyl groups include indolinyl, isoindolinyl, dihydrobenzofuranyl, dihydrobenzothienyl, tetrahydrobenzo-thienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, and 1,2,3,4-tetrahydro-1,6-naphthyridinyl.

The term “heteroaryl” means 4-8 membered monocyclic, 8-12 membered bicyclic, or 11-14 membered tricyclic aromatic ring systems having 1-4 heteroatoms (if monocyclic), ₁₋₆heteroatoms (if bicyclic), or 1-9 heteroatoms (if tricyclic) that are each independently selected from N, O, or S (e.g., carbon atoms and _{1-3, 1}-6, or 1-9 heteroatoms independently selected from N, O, or S, if monocyclic, bicyclic, or tricyclic, respectively). Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

The term “halogen” means a monovalent substituent that is fluorine (fluoro), chlorine (chloro), bromine (bromo), or iodine (iodo). The prefix “halo” means a group including one or more of a fluoro, chloro, bromo, or iodo substituent instead of a hydrogen atom. A combination of halo groups (e.g., bromo and fluoro), or only fluoro groups may be present. For example, the term “haloalkyl” refers to an alkyl group substituted with one or more halogens. As used herein, “substituted C1_8 haloalkyl” refers to a C1-8 alkyl group substituted with at least one halogen, and is further substituted with one or more other substituent groups that are not halogens. It is to be understood that substitution of a group with a halogen atom is not to be considered a heteroatom-containing group, because a halogen atom does not replace a carbon atom.

The term “fluorinated” means having one or more fluorine atoms incorporated into a group instead of hydrogen. For example, where a C₁₋₁₈ fluoroalkyl group is indicated, the fluoroalkyl group can include one or more fluorine atoms, for example, a single fluorine atom, two fluorine atoms (e.g., as a 1,1-difluoroethyl group), three fluorine atoms (e.g., as a 2,2,2-trifluoroethyl group), or fluorine atoms at each valence of carbon (e.g., as a perfluorinated group such as —CF₃, —C₂F₅, —C₃F₇, or —C₄F₉). A “substituted fluoroalkyl group” shall be understood to mean a fluoroalkyl group that is further substituted by at least one additional substituent group that does not contain fluorine atoms.

Each of the foregoing substituent groups optionally may be substituted unless expressly provided otherwise. The term “optionally substituted” refers to being substituted or unsubstituted. “Substituted” means that at least one hydrogen atom of the chemical structure or group is replaced with another terminal substituent group that is typically monovalent, provided that the designated atom's normal valence is not exceeded. When the substituent is oxo (i.e., O), then two geminal hydrogen atoms on the carbon atom are replaced with the terminal oxo group. It is further noted that the oxo group is bonded to carbon via a double bond to form a carbonyl (C═O), where the carbonyl group is represented herein as —C(O)—. Combinations of substituents or variables are permissible. Exemplary substituent groups that may be present on a “substituted” position include, but are not limited to, nitro (—NO₂), cyano (—CN), hydroxyl (—OH), oxo (O), amino (—NH2), mono- or di-(C₁₋₆)alkylamino, alkanoyl (such as a C₂₋₆ alkanoyl group such as acyl), formyl (—C(O))H), carboxylic acid or an alkali metal or ammonium salt thereof; esters (including acrylates, methacrylates, and lactones) such as C₂₋₆ alkyl esters (—C(O))O-alkyl or —OC(O)—alkyl) and C₇₋₁₃ aryl esters (—C(O))O-aryl or —OC(O)—aryl); amido (—C(O))NR₂ wherein R is hydrogen or C₁₋₆ alkyl), carboxamido (—CH₂C(O)NR₂ wherein R is hydrogen or C₁₋₆ alkyl), halogen, thiol (—SH), C₁₋₆ alkylthio (—S-alkyl), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₂₋₁₈ heterocycloalkenyl, C₆₋₁₂ aryl having at least one aromatic ring (e.g., phenyl, biphenyl, naphthyl, or the like, each ring either substituted or unsubstituted aromatic), C₇₋₁₉ arylalkyl having 1 to 3 separate or fused rings and from 6 to 18 ring carbon atoms, arylalkoxy having 1 to 3 separate or fused rings and from 6 to 18 ring carbon atoms, C₇₋₁₂ alkylaryl, C₃₋₁₂ heterocycloalkyl, C₃₋₁₂ heteroaryl, C₁₋₆ alkyl sulfonyl (—S(O)₂-alkyl), C₆₋₁₂ arylsulfonyl (—S(O)₂-aryl), or tosyl (CH₃C₆H₄SO₂—). When a group is substituted, the indicated number of carbon atoms is the total number of carbon atoms in the group, excluding those of any substituents. For example, the group —CH₂CH₂CN is a cyano-substituted C₂ alkyl group.

As used herein, when a definition is not otherwise provided, a “divalent linking group” refers to a divalent group including one or more of —O—, —S—, —Te—, —Se—, —C(O))—, C(O)O—, —N(R′)—, —C(O))N(R′)—, —S(O)—, —S(O)₂—, —C(S)—, —C(Te)—, —C(Se)—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein each R′ is independently hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. Typically, the divalent linking group includes one or more of —O—, —S—, —C(O))—, —N(R′)—, —S(O)—, —S(O)₂—, substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, or a combination thereof, wherein R′ is hydrogen, substituted or unsubstituted C₁₋₂₀ alkyl, substituted or unsubstituted C₁₋₂₀ heteroalkyl, substituted or unsubstituted C₆₋₃₀ aryl, or substituted or unsubstituted C₃₋₃₀ heteroaryl. More typically, the divalent linking group includes at least one of —O—, —C(O))—, —C(O))O—, —N(R′)—, —C(O))N(R′)—, substituted or unsubstituted C₁₋₁₀ alkylene, substituted or unsubstituted C₃₋₁₀ cycloalkylene, substituted or unsubstituted C₃₋₁₀ heterocycloalkylene, substituted or unsubstituted C₆₋₁₀ arylene, substituted or unsubstituted C₃₋₁₀ heteroarylene, or a combination thereof, wherein R is hydrogen, substituted or unsubstituted C₁₋₁₀ alkyl, substituted or unsubstituted C₁₋₁₀ heteroalkyl, substituted or unsubstituted C₆₋₁₀ aryl, or substituted or unsubstituted C₃₋₁₀ heteroaryl.

As used herein, an “acid-labile group” refers to a group in which a bond is cleaved by the action of an acid, optionally and typically with thermal treatment, resulting in formation of a polar group, such as a carboxylic acid or alcohol group, being formed on the polymer, and optionally and typically with a moiety connected to the cleaved bond becoming disconnected from the polymer. In other systems, a non-polymeric compound may include an acid-labile group that may be cleaved by the action of an acid, resulting in formation of a polar group, such as a carboxylic acid or alcohol group on a cleaved portion of the non-polymeric compound. Such acid is typically a photo-generated acid with bond cleavage occurring during post-exposure baking (PEB); however, embodiments are not limited thereto, and, for example, such acid may be thermally generated. Suitable acid-labile groups include, for example: tertiary alkyl ester groups, secondary or tertiary aryl ester groups, secondary or tertiary ester groups having a combination of alkyl and aryl groups, tertiary alkoxy groups, acetal groups, or ketal groups. Acid-labile groups are also commonly referred to in the art as “acid-cleavable groups,” “acid-cleavable protecting groups,” “acid-labile protecting groups,” “acid-leaving groups,” “acid-decomposable groups,” and “acid-sensitive groups.”

The term “unsaturated bond” refers to a double or triple bond. The term “unsaturated” or “partially unsaturated” refers to a moiety that includes at least one double or triple bond. The term “saturated” refers to a moiety that does not contain a double or triple bond, i.e., the moiety only contains single bonds.

As used herein, the term “(meth)acrylic” includes both acrylic and methacrylic species (i.e., acrylic and methacrylic monomers), and the term “(meth)acrylate” includes both acrylate and methacrylate species (i.e., acrylate and methacrylate monomers).

Iodine has a remarkably high absorption cross section to EUV radiation (i.e., 13.5 nm). However, functionalization with iodine increases hydrophobicity of organic molecules. For instance, the generation of iodine-rich by-product after post exposure bake step might not allow complete or clean development of the expose area by an alkali developer. To overcome these difficulties, the present invention provides an acid-labile compound comprising one or more iodine substituents and one or more carboxylic acid functionalities that are protected with an acid labile group. The acid-labile can be used to provide photoresist polymers having an increased resist absorption at EUV exposure wavelength without compromising the unexposed film thickness. Acid catalyzed deprotection of the repeat units that are derive from the acid-labile compounds results in iodine-containing structural units that are hydrophilic and soluble in alkali developer. In addition, upon deprotection, the iodine-containing structural units remain attached to the backbone of the polymer, and thus the deprotection products remaining after post-exposure bake are free of iodine.

The inventive iodine-containing compound includes an aromatic group or a heteroaromatic group, wherein the aromatic group or the heteroaromatic group comprises a first substituent group comprising an ethylenically unsaturated double bond, a second substituent group that is an iodine atom, and a third substituent comprising an acid-labile group. The first substituent, the second substituent, and the third substituent are each bonded to a different carbon atom of the aromatic group or heteroaromatic group.

As used herein, “an aromatic group or a heteroaromatic group” refers to a monocyclic or polycyclic C₆₋₆₀ aromatic group or a monocyclic or polycyclic C₃₋₆₀ heteroaromatic group. When the C₆₋₆₀ aromatic group is polycyclic, the ring or ring groups can be fused (such as naphthyl or the like), or directly linked (such as biaryls, biphenyl, or the like). In an embodiment, the polycyclic aromatic group may include a combination of fused and directly linked ring or ring groups (such as binaphthyl or the like). When the C₃₋₆₀ heteroaromatic group is polycyclic, the ring or ring groups can be fused, directly linked, or a combination of fused and directly linked ring or ring groups.

The first substituent group of the aromatic group or the heteroaromatic group includes an ethylenically unsaturated double bond. As used herein, an “ethylenically unsaturated double bond” refers to a vinyl-containing polymerizable group, and typically may be selected from substituted or unsubstituted C₂₋₂₀ alkenyl, substituted or unsubstituted norbornyl, substituted or unsubstituted (meth)acrylic, substituted or unsubstituted vinyl ether, substituted or unsubstituted vinyl ketone, substituted or unsubstituted vinyl ester, or substituted or unsubstituted vinyl aromatic.

The second substituent group of the aromatic group or the heteroaromatic group is an iodine atom (—I). It is to be understood that the second substituent group may include one or more iodine atoms. In some aspects, the second substituent group can include from 1 to 9 iodine atoms, or from 1 to 5 iodine atoms, or from 1 to 3 iodine atoms, or 1 or 2 iodine atoms. In other words, the iodine-containing compound may include from 1 to 9 iodine atoms, or from 1 to 5 iodine atoms, or from 1 to 3 iodine atoms, or 1 or 2 iodine atoms.

The third substituent group of the aromatic group or the heteroaromatic group includes an acid-labile group. It is to be understood that the third substituent group may include one acid-labile group, or may include 2 or more acid-labile groups that are the same or different from each other. In some aspects, the third substituent group may contain from 1 to 5 different acid-labile groups, or from 1 to 3 different acid-labile groups, or 2 different acid-labile groups, or a single acid labile group (i.e., one acid labile group). In other words, the iodine-containing compound may include from 1 to 5 different acid-labile groups, or from 1 to 3 different acid-labile groups, or 2 different acid-labile groups, or a single acid labile group (i.e., one acid labile group).

Suitable acid-labile groups of the third substituent group include, for example, one or more of tertiary alkyl ester groups, secondary or tertiary aryl ester groups, secondary or tertiary ester groups having a combination of alkyl and aryl groups, tertiary alkoxy groups, acetal groups, or ketal groups.

In some embodiments, the number of second substituent groups and the number of third substituent groups, when combined, equals 10 or less, or is from 2 to 6, or is from 2 to 4. In other words, the iodine-containing compound may include, in total, 10 or fewer iodine atoms and acid-labile groups combined, or, for example, a total number of iodine atoms and acid-labile groups is from 2 to 6, more typically from 2 to 4.

In some aspects, the first substituent group does not comprise an acid-labile group or an acid leaving group. In other words, in some aspects the iodine-containing compound includes a polymerizable group (i.e., an ethylenically unsaturated double bond) that is not a tertiary alkyl ester group, a secondary or tertiary aryl ester group, a secondary or tertiary ester group having a combination of alkyl and aryl groups, a tertiary alkoxy group, an acetal group, or a ketal group. For example, the first substituent group of the inventive compound may be (meth)acrylic or vinyl (e.g., substituted or unsubstituted C₂₋₁₂ alkenyl).

In some aspects, the compound may be represented by Formula (1):

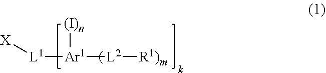

In Formula (1), Ar¹ is an aromatic group or a heteroaromatic group, the first substituent group is represented by -L¹-X, the second substituent group is represented by —I, and the third substituent group is represented by -L²-R¹.

Ar¹ is C₆₋₃₀ aryl or C₃₋₃₀ heteroaryl, and each optionally may be further substituted with one or more of substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl.

It is to be understood that “further substituted” means the C₆₋₃₀ aryl group or the C₃₋₃₀ heteroaryl group is substituted as required by Formula (1) with at least the first substituent group (-L¹-X), the second substituent group (I)., and the third substituent group (-L²-R¹), and the C₆₋₃₀ aryl group or the C₃₋₃₀ heteroaryl group optionally may be further substituted with one or more other substituent groups that are different from the first substituent group, the second substituent group, and the third substituent group. Typically, Ar¹ is C₆₋₃₀ aryl, and optionally may be further substituted with one or more of substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₁₋₃₀ heterocycloalkyl, or a combination thereof.

In Formula (1), X is a polymerizable group comprising the ethylenically unsaturated double bond. Preferably, X is (meth)acrylic or substituted or unsubstituted C₂₋₁₂ alkenyl.

In Formula (1), L¹ is a single bond or a divalent linking group. For example, L¹ may be a divalent linking group including one or more of substituted or unsubstituted C₁₋₃₀ alkylene, substituted or unsubstituted C₃₋₃₀ cycloalkylene, substituted or unsubstituted C₁₋₃₀ heterocycloalkylene, substituted or unsubstituted C₆₋₃₀ arylene, substituted or unsubstituted C₁₋₃₀ heteroarylene, —O—, —C(O))—, —C(O))O—, —C(O)NR^{1a}—, or —N(R^{1b})—, wherein R^{1a} and R^{1b} are each independently hydrogen or C₁₋₆ alkyl.

In Formula (1), the first substituent group may be defined by the moiety -L¹-X, wherein L¹ is a single bond or a divalent linking group and X is the polymerizable group comprising an ethylenically unsaturated double bond. Typically, X is substituted or unsubstituted C₂₋₂₀ alkenyl, substituted or unsubstituted norbornyl, or substituted or unsubstituted (meth)acrylic. For example, when the first substituent group comprises a substituted or unsubstituted vinyl aromatic group, in the first substituent group -L¹-X, L¹ is a substituted or unsubstituted divalent C₇ alkylaryl group (or C7 arylalkyl group) and X is C₂ alkenyl.

In Formula (1), n represents the number of iodine atoms that are bonded directly to the aromatic group or the heteroaromatic group, and is an integer greater than or equal to 1. In some aspects, n is an integer from 1 to 9, or an integer from 1 to 7, or an integer from 1 to 5, or an integer from 1 to 4, or an integer from 1 to 3, or 1 or 2. Preferably, n is 1 or 2.

In Formula (1), m represents the number of third substituent groups, where the third substituent groups may be defined by the moiety -L²-R¹, and is an integer greater than or equal to 1. In some aspects, m is preferably an integer from 1 to 5, or an integer from 1 to 4, or an integer from 1 to 3, or 1 or 2. Preferably, m is an integer from 1 to 3.

In Formula (1), the sum of n and m (n+m) is an integer of 10 or less. For example, the sum of n and m (n+m) may be an integer from 2 to 8, or from 2 to 6, or from 2 to 4. Preferably, the sum of n and m (n+m) is an integer from 2 to 4.

In Formula (1), k is an integer from 1 to 5. Typically, k is 1.

In Formula (1), R¹ comprises an acid-labile group. Exemplary acid-labile groups include tertiary alkyl ester groups, secondary or tertiary aryl ester groups, secondary or tertiary ester groups having a combination of alkyl and aryl groups, tertiary alkoxy groups, acetal groups, or ketal groups.

In some aspects, R¹ may have a structure represented by one of Formula (2) or Formula (3):

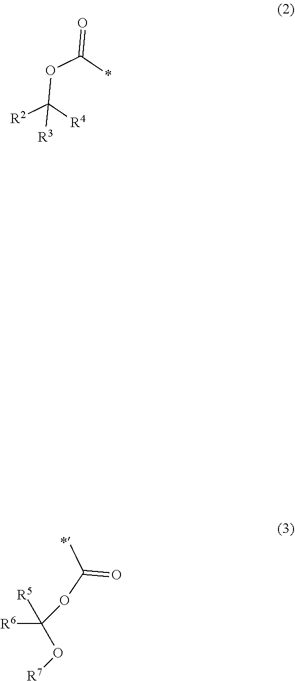

In Formula (2), R² to R⁴ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₂₋₂₀ heteroaryl, provided that no more than one selected from R² to R⁴ is hydrogen, and provided that if one of R² to R⁴ is hydrogen, then at least one of the others from R² to R⁴ is substituted or unsubstituted C₆₋₂₀ aryl or substituted or unsubstituted C₃₋₂₀ heteroaryl. Each of R² to R⁴ may optionally further include a divalent linking group as part of its structure. For example, each of R² to R⁴ may further comprise as part of its structure one or more groups selected from —O—, —C(O))—, —C(O))O—, —S-, —S(O)₂—, —N(R^{2a})—, or —C(O))N(R^{2b})— wherein R^{2a} and R^{2b} are each independently hydrogen, substituted or unsubstituted C₁₋₂₀ alkyl, substituted or unsubstituted C₃₋₂₀ cycloalkyl, or substituted or unsubstituted C₃₋₂₀ heterocycloalkyl. Typically, R² to R⁴ are each independently hydrogen, substituted or unsubstituted C₁₋₁₀ alkyl, substituted or unsubstituted C₃₋₈ cycloalkyl, or substituted or unsubstituted C₆₋₁₄ aryl.

In Formula (3), R⁵ and R⁶ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl. Each of R⁵ and R⁶ may optionally further include a divalent linking group as part of its structure. For example, each of R⁵ and R⁶ may further comprise as part of its structure one or more groups selected from —O—, —C(O))—, —C(O)O—, —S—, —S(O)₂—, —N(R^{3a})—, or —C(O))N(R^{3b})—, wherein R^{3a} and R^{3b} are each independently hydrogen, substituted or unsubstituted C₁₋₂₀ alkyl, substituted or unsubstituted C₃₋₂₀ cycloalkyl, or substituted or unsubstituted C₃₋₂₀ heterocycloalkyl. Typically, R⁵ and R⁶ are each independently hydrogen, or substituted or unsubstituted C₁₋₁₀ alkyl.

In Formula (3), R⁷ is substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₃₋₂₀ heteroaryl. R⁷ optionally may further comprise a divalent linking group as part of its structure. Typically, R⁷ may be substituted or unsubstituted C₁₋₁₀ alkyl, substituted or unsubstituted C₃₋₈ cycloalkyl, or substituted or unsubstituted C₆₋₁₄ aryl.

In Formula (2), any two of R², R³, or R⁴ together optionally may form a ring via a single bond or a divalent linking group, wherein the ring is substituted or unsubstituted. In Formula (3), R⁵ and R⁶ together optionally may form a ring via a single bond or a divalent linking group, wherein the ring is substituted or unsubstituted. In Formula (3), any one or more of R⁵ or R⁶ together with R⁷ optionally may form a ring via a single bond or a divalent linking group, wherein the ring is substituted or unsubstituted.

In Formulae (2) and (3), * and *' each indicate a binding site to L². It is to be understood that when L² is a single bond, the corresponding * or *' indicates a binding site to Ar¹.

In one or more embodiments, in Formulae (1), (2), and (3), n is 1 or 2; X is (meth)acrylic or substituted or unsubstituted C₂₋₁₂ alkenyl; L¹ is a single bond; L² is a single bond or —C(O))OC(X¹X²)—, wherein X¹ and X² are each independently hydrogen, fluorine, unsubstituted C₁₋₆alkyl, C₁₋₆-fluoroalkyl, unsubstituted C₃₋₆ cycloalkyl, or C₃₋₆- fluorocycloalkyl, and typically, X¹ and X² are hydrogen; and Ar¹ is C₆₋₁₀aryl, optionally further substituted with one or more of substituted or unsubstituted C₁₋₁₀ alkyl, substituted or unsubstituted C₁₋₁₀ heteroalkyl, substituted or unsubstituted C₃₋₁₀ cycloalkyl, or substituted or unsubstituted C₃₋₁₀ heterocycloalkyl. In this embodiment, R² to R⁴ are each independently hydrogen, substituted or unsubstituted C₁₋₁₀ alkyl, substituted or unsubstituted C³⁻⁸ cycloalkyl, or substituted or unsubstituted C₆₋₁₄ aryl, provided that no more than one selected from R² to R⁴ is hydrogen, and provided that if one of R² to R⁴ is hydrogen, then at least one of the others from R² to R⁴ is substituted or unsubstituted C⁶⁻¹⁴ aryl; any two of R² to R⁴ together optionally form a ring via a single bond or a divalent linking group, wherein the ring is substituted or unsubstituted; R⁵ and R⁶ are each independently hydrogen, or substituted or unsubstituted C₁₋₁₀ alkyl; and R⁷ is substituted or unsubstituted C₁₋₁₀ alkyl, substituted or unsubstituted C₃₋₈ cycloalkyl, or substituted or unsubstituted C₆₋₁₄ aryl.

Exemplary iodine-containing compounds may include the following:

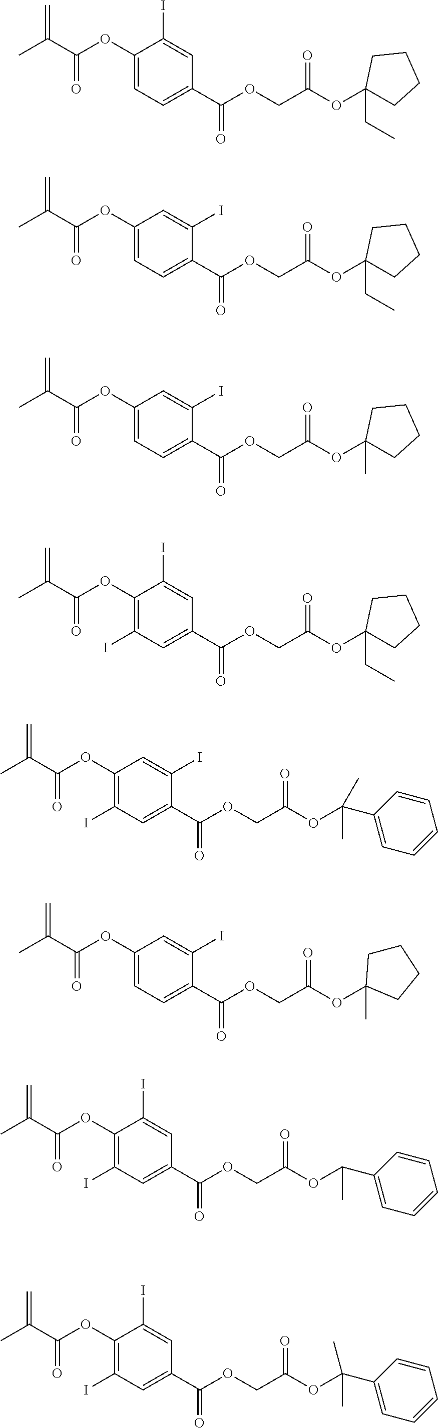

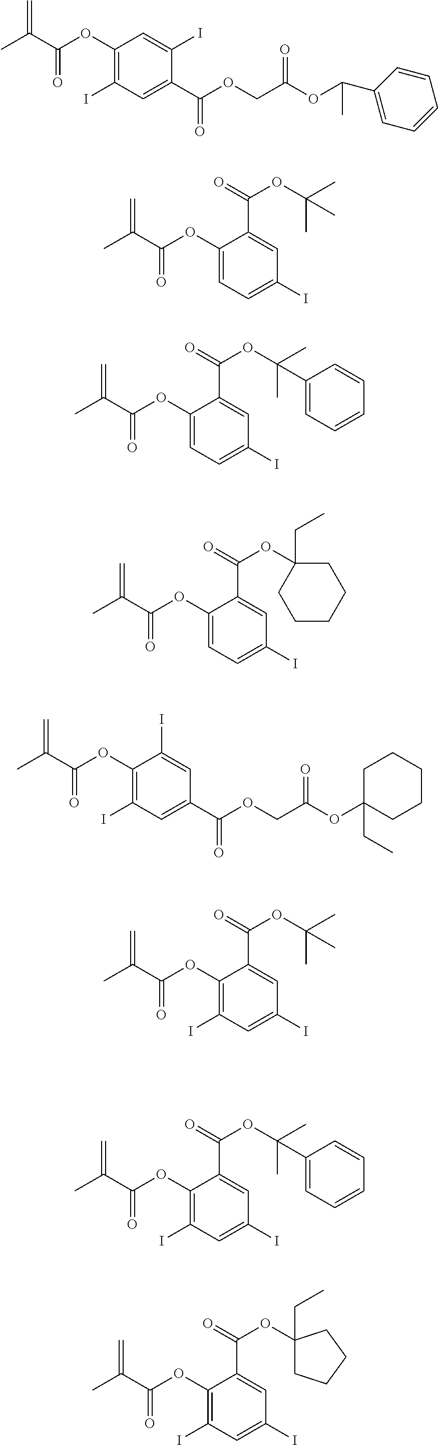

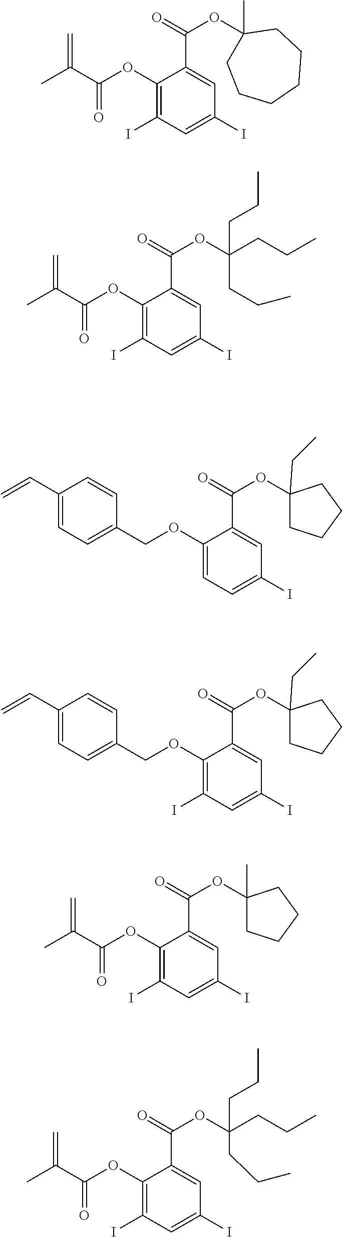

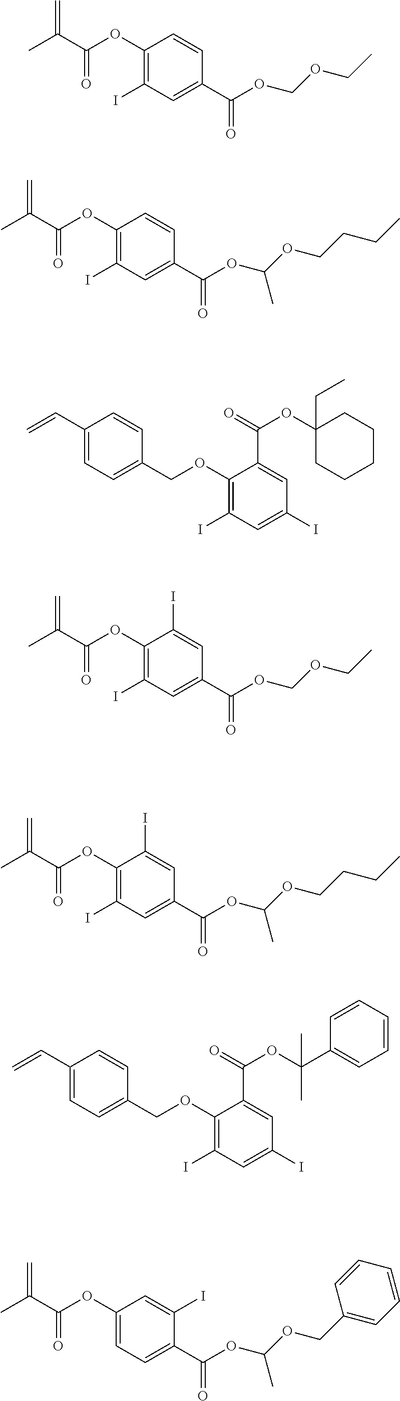

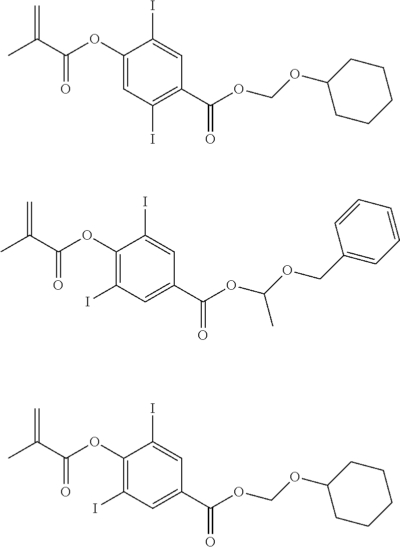 [SMILES: C=C(C)C(=O)Oc1cc(I)c(C(=O)OCOC2CCCCC2)cc1I ; C=C(C)C(=O)Oc1c(I)cc(C(=O)OC(C)OCc2ccccc2)cc1I ; C=C(C)C(=O)Oc1c(I)cc(C(=O)OCOC2CCCCC2)cc1I]

Also provided is a polymer comprising a first repeating unit derived from the inventive compound described herein. As would be understood in the art, the inventive compound may be used a monomer used to prepare the polymer, where the resulting polymer includes a first repeating unit that is derived from the inventive compound. The inventive compound is also referred to herein as the inventive monomer compound, or simply as the “monomer compound” for the sake of convenience. The inventive compound may also be referred to herein as “a first monomer.”

The first repeating unit derived from the inventive monomer compound is typically present in the polymer in an amount from 0.1 to 50 mole percent (mol %), more typically from 1 to 25 mol %, still more typically from 5 to 15 mol %, based on total repeating units in the polymer.

When the polymer includes a first repeating unit derived from the inventive monomer compound, the structural unit includes an iodine-containing aromatic or heteroaromatic group portion that is not acid-cleavable from the polymer backbone. The present inventors have surprisingly discovered the when the polymer including the first repeating unit is exposed to irradiation (and subsequent PEB), the iodine-substituted aromatic group remains attached to the polymer backbone and thus enhances EUV absorption.

In some aspects, the polymer may further include a repeating unit comprising an acid-labile group, in other words, the polymer may comprise a first repeating unit derived from the inventive monomer compound that includes a first acid-labile group (e.g., the third substituent group) and a second repeating unit comprising a second acid labile group, where the second acid labile group is different from the first acid-labile group.

In one or more embodiments, the polymer may include an acid-labile repeating unit derived from a monomer represented by one or more of Formulae (4), (5), (6), (7), or (8):

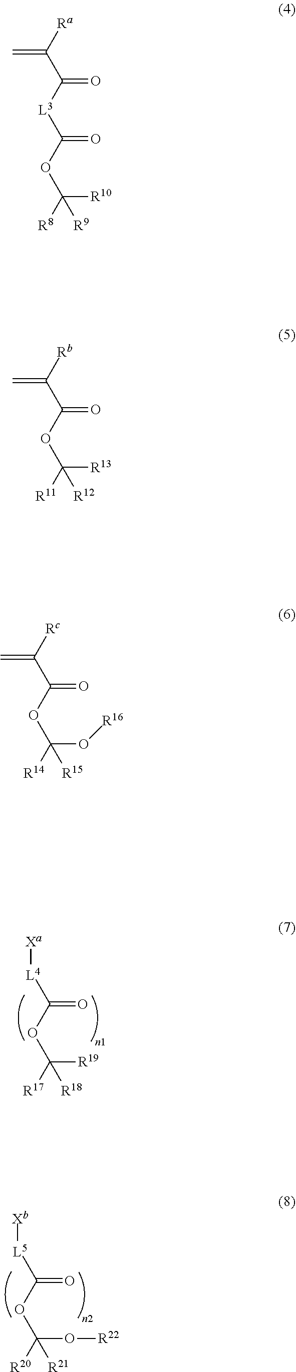

In Formulae (4), (5) and (6), Ra, Rb, and W may each independently be hydrogen, fluorine, cyano, or substituted or unsubstituted C₁₋₁₀ alkyl. Preferably, Ra, Rb, and W may each independently be hydrogen, fluorine, or substituted or unsubstituted C1_5 alkyl, typically methyl.

In Formula (4), L³ is a divalent linking group. For example, L³ may include 1 to 10 carbon atoms and at least one heteroatom. In a typical example, L¹ may be —OCH₂—, —OCH₂CH₂O—, or —N(R^{4a})—, wherein R^{4a} is hydrogen or C₁₋₆ alkyl.

In Formulae (4) and (5), R⁸ to R¹³ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₃₋₂₀ heteroaryl, provided that no more than one of R⁸ to R¹⁰ may be hydrogen, and that no more than one of R″ to R¹³ may be hydrogen, and provided that if one of R⁸ to R¹⁰ is hydrogen, then at least one of the others from R⁸ to R¹⁰ is substituted or unsubstituted C₆₋₂₀ aryl or substituted or unsubstituted C₃₋₂₀ heteroaryl, and if one of R¹¹ to R¹³ is hydrogen, then at least one of the others from R¹¹ to R¹³ is substituted or unsubstituted C₆₋₂₀ aryl or substituted or unsubstituted C₃₋₂₀ heteroaryl. Preferably, R⁸ to R¹³ are each independently substituted or unsubstituted C₁₋₆ alkyl or substituted or unsubstituted C₃₋₁₀ cycloalkyl. Each of R⁸ to R¹³ may optionally further comprise a divalent linking group as part of their structure.

For example, any one or more of R⁸ to R¹³ may be independently a group of the formula —CH₂C(O)CH₍₃₋ₙ₎Yₙ, or —CH₂C(O)OCH₍₃₋ₙ₎Yₙ, where each Y is independently substituted or unsubstituted C₃₋₁₀ heterocycloalkyl and n is 1 or 2. For example, each Y may be independently substituted or unsubstituted C₃₋₁₀ heterocycloalkyl including a group of the formula —O(C^{a1})(C^{a2})O—, wherein C^{a1} and C^{a2} are each independently hydrogen or substituted or unsubstituted alkyl, and where C^{a1} and C^{a2} together optionally form a ring.

In Formula (4), any two of R⁸ to R¹⁰ together optionally may form a ring, which may further include a divalent linking group as part of its structure, and wherein the ring may be substituted or unsubstituted. In Formula (5), any two of R¹¹ to R¹³ together optionally may form a ring, which may further include a divalent linking group as part of its structure, wherein the ring group may be substituted or unsubstituted.

In Formulae (6) and (8), R¹⁴, R¹⁵, R²⁰, and R²¹ each independently may be hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₃₋₂₀ heteroaryl; and R¹⁶ and R²² are each independently substituted or unsubstituted C₁₋₂₀ alkyl, substituted or unsubstituted C₃₋₂₀ cycloalkyl, or substituted or unsubstituted C₃₋₂₀ heterocycloalkyl. Preferably, R¹⁴, R¹⁵, R²⁰, and R²¹ each independently may be hydrogen, substituted or unsubstituted C₁₋₂₀ alkyl, substituted or unsubstituted C₃₋₂₀ cycloalkyl, or substituted or unsubstituted C₃₋₂₀ heterocycloalkyl. Each of R¹⁴, R¹⁵, R²⁰, and R²¹ may optionally further comprise a divalent linking group as part of their structure.

In Formula (4), any two of R¹⁴ to R¹⁶ together optionally may form a ring, which may further include a divalent linking group as part of its structure, wherein the ring group may be substituted or unsubstituted.

In Formula (7), R¹⁷ to R¹⁹ may be each independently be substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₃₋₂₀ heteroaryl, provided that no more than one of R¹⁷ to R¹⁹ may be hydrogen and provided that if one of R¹⁷ to R¹⁹ is hydrogen, then at least one of the others from R¹⁷ to R¹⁹ is substituted or unsubstituted C₆₋₂₀ aryl or substituted or unsubstituted C₃₋₂₀ heteroaryl. Each of R¹⁷ to R¹⁹ may optionally further comprise a divalent linking group as part of their structure.

For example, any one or more of R¹⁷ to R¹⁹ may be independently a group of the formula —CH₂C(O)CH₍₃₋ₙ₎Yₙ, or —CH₂C(O)OCH₍₃₋ₙ₎Yₙ, where each Y is independently substituted or unsubstituted C₃₋₁₀ heterocycloalkyl and n is 1 or 2. For example, each Y may be independently substituted or unsubstituted C₃₋₁₀ heterocycloalkyl including a group of the formula —O(C^{a1})(C^{a2})O—, wherein C^{a1} and C^{a2} are each independently hydrogen or substituted or unsubstituted alkyl, and where C^{a1} and C^{a2} together optionally form a ring.

In Formula (7), any two of R¹⁷ to R¹⁹ together optionally form a ring, which may further include a divalent linking group as part of its structure, wherein the ring group may be substituted or unsubstituted.

In Formulae (7) and (8), X^{a} and X^{b} are each independently a polymerizable group comprising an ethylenically unsaturated double bond, preferably (meth)acrylate or C₂ alkenyl.

In Formulae (7) and (8), L⁴ and L⁵ are each independently a single bond or a divalent linking group, provided that L⁴ is not a single bond when X^{a} is C₂ alkenyl and that L⁵ is not a single bond when X^{b} is C₂ alkenyl. Preferably, L⁴ and L⁵ are each independently substituted or unsubstituted C₆-₃₀ arylene or substituted or unsubstituted C₆₋₃₀ cycloalkylene. In Formulae (7) and (8), n1 is 0 or 1 and n2 is 0 or 1. It is to be understood that when n1 is 0, the L⁴ group is connected directly to the oxygen atom. It is to be understood that when n2 is 0, the L⁵ group is connected directly to the oxygen atom.

In Formula (8), any two of R¹⁸ to R²° together optionally may form a ring, which may further include a divalent linking group as part of its structure, wherein the ring group may be substituted or unsubstituted.

In some aspects, each of R⁸ to R²² optionally may further include as part of their structure one or more divalent linking groups selected from —O—, —C(O))—, —C(O))O—, —S—, —S(O)₂—, —N(R′)—, or —C(O))N(R′)—, wherein R′ may be hydrogen, substituted or unsubstituted C₁₋₂₀ alkyl, substituted or unsubstituted C₃₋₂₀ cycloalkyl, or substituted or unsubstituted C₃₋₂₀ heterocycloalkyl.

In some aspects, in a repeating unit comprising an acid-labile group, the acid-labile group may be a tertiary alkyl ester. For example, a repeating unit comprising a tertiary alkyl ester group may be derived from one or more monomers of Formulae (4), (5), or (7), wherein none of R⁸ to R¹³ or R¹⁷ to R¹⁹ is hydrogen, and n1 is 1. In one or more embodiments, the polymer further comprises a second repeating unit comprising a tertiary alkyl ester group.

Exemplary monomers of Formula (4) include one or more of the following:

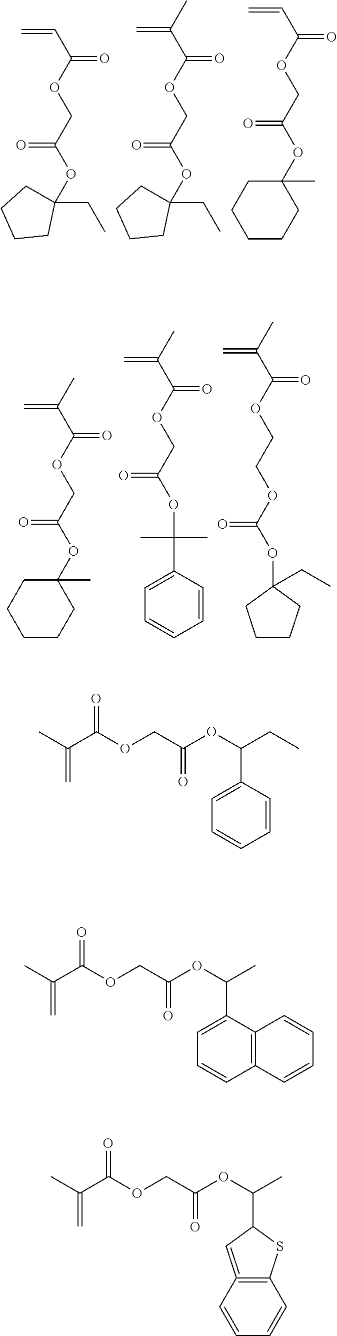

Exemplary monomers of Formula (5) include one or more of the following:

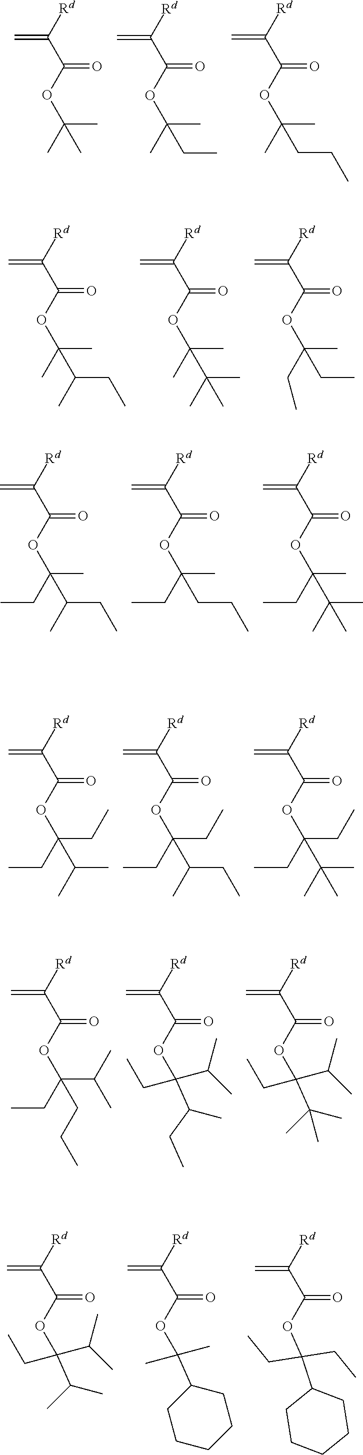

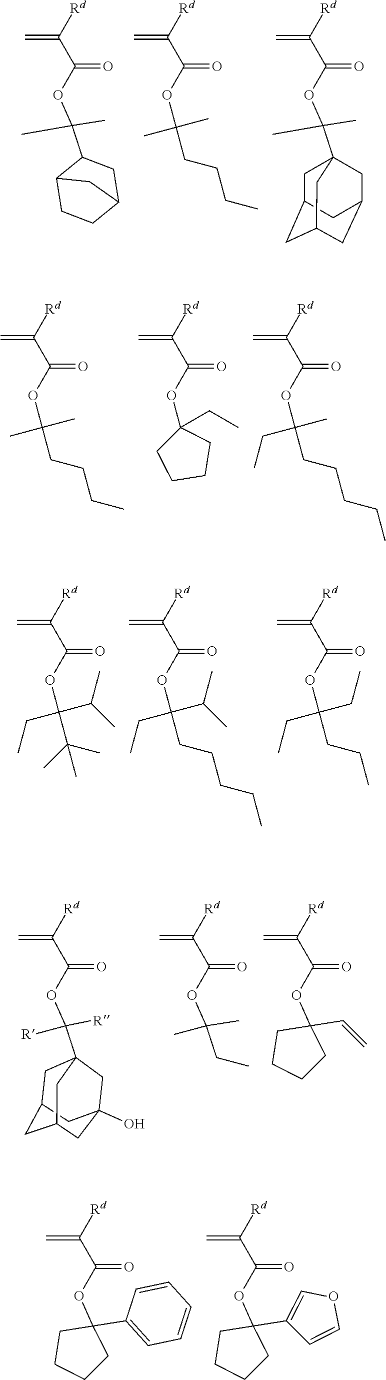

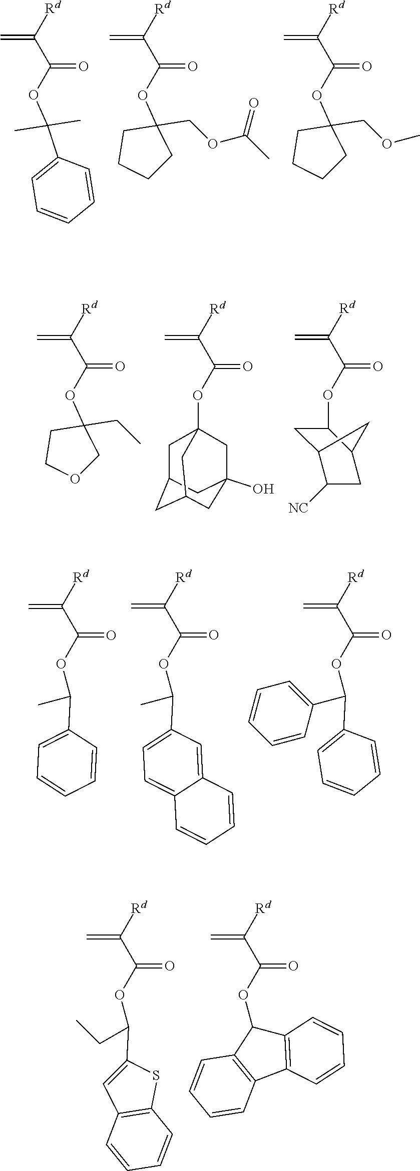

wherein R^{d} is as defined herein for R^{b} in Formula (3); and R′ and R″ are each independently substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C2_20 alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₃₋₂₀ heteroaryl.

Exemplary monomers of Formula (6) include one or more of the following:

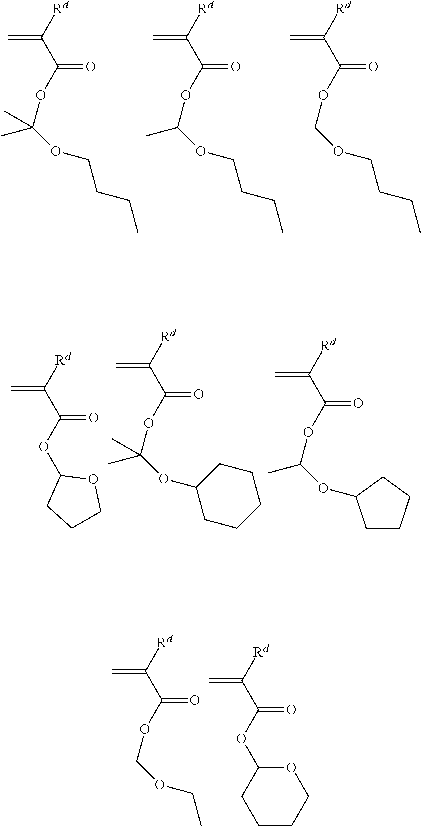

wherein R^{d} is as defined above for R^{c}.

Exemplary monomers of Formula (7) include one or more of the following:

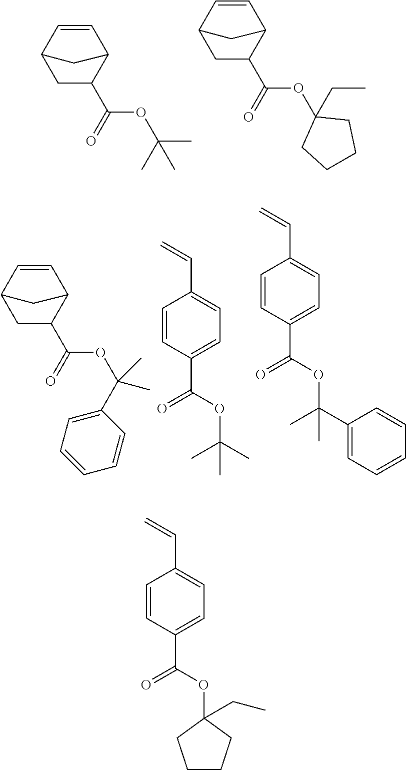

Exemplary monomers of Formula (8) include one or more of the following:

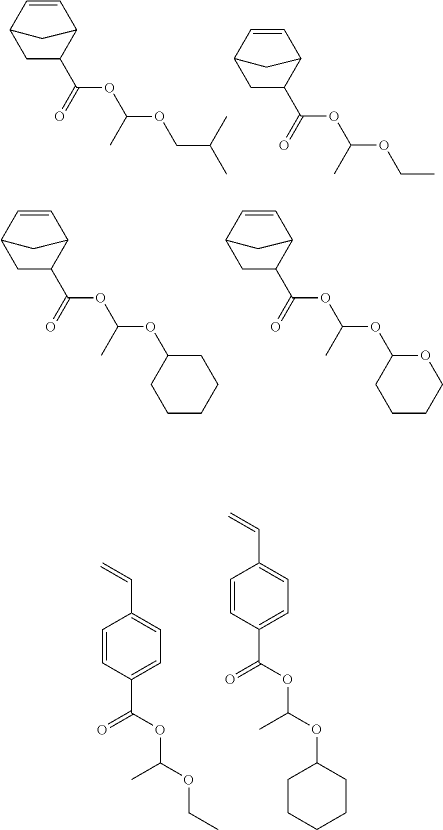

In some aspects, the polymer may have an acid-labile repeating unit that is derived from one or more monomers having a cyclic acetal or cyclic ketal group, for example, having one or more of the following structures:

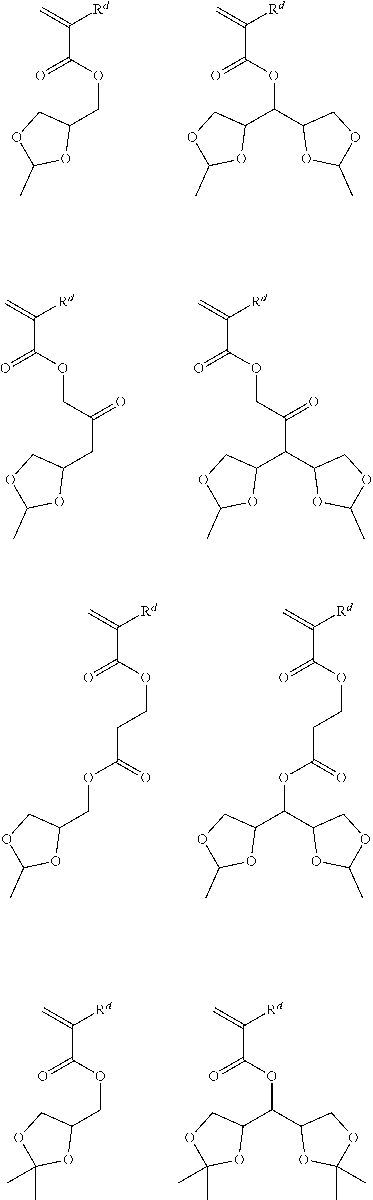

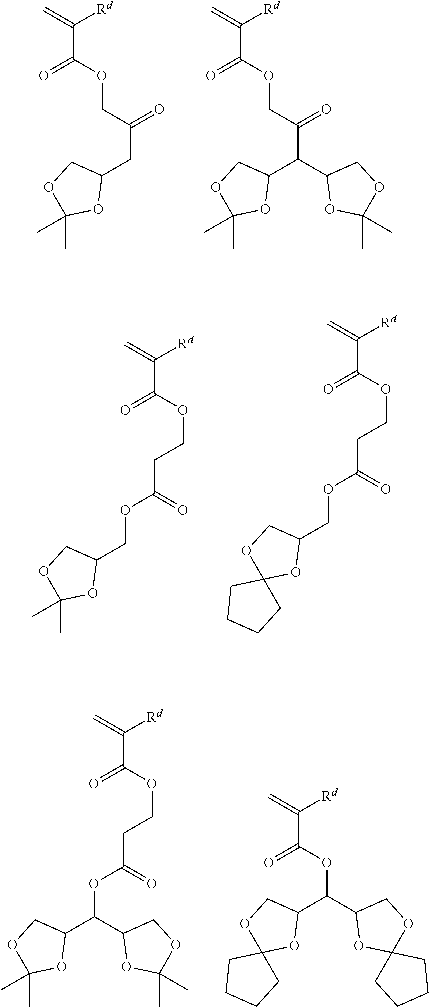

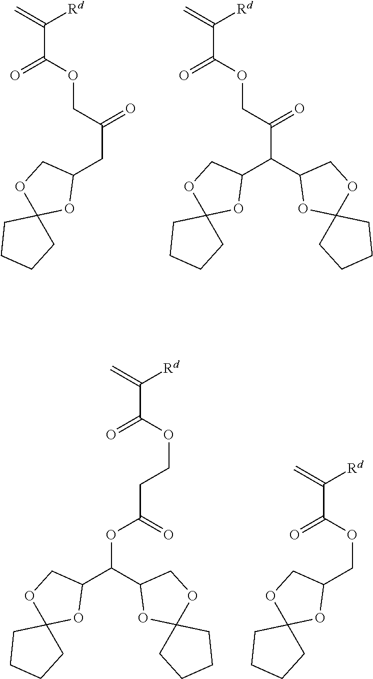

wherein R^{d} is as defined above for R^{a}.

In some aspects, the polymer may have a repeating unit having an acid-labile group that comprises a tertiary alkoxy group, for example, one or more monomers of the following:

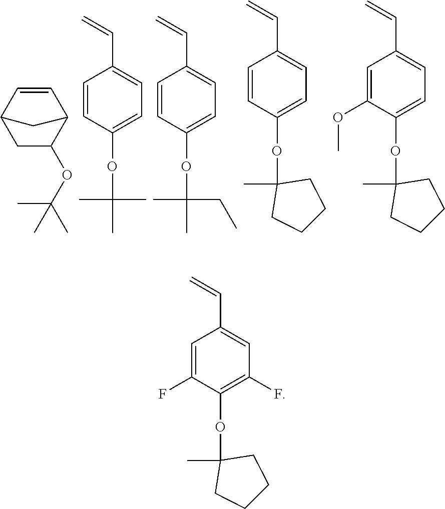

The second repeating unit having an acid-labile group, and that is different from first repeating unit, is typically present in the polymer in an amount from 25 to 65 mol %, more typically from 30 to 50 mol %, still more typically from 30 to 45 mol %, based on total repeating units in the polymer.

In some aspects, the polymer may further include a repeating unit (e.g., “a third repeating unit”) comprising a polar group, where the polar group is pendant to the backbone of the polymer. For example, the polar group can be a lactone group, a hydroxy aryl group, a fluoroalcohol group, or a combination thereof.

In one or more embodiments, the polymer may further include a third repeating unit derived from one or more lactone-containing monomers of Formula (9):

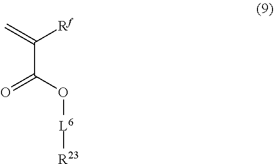

wherein R^{f} is hydrogen, fluorine, cyano, or substituted or unsubstituted C₁₋₁₀ alkyl.

In Formula (9), L⁶ is a single bond or a divalent linking group. Exemplary divalent linking groups for L⁶ include one or more of substituted or unsubstituted C₁₋₃₀ alkylene, substituted or unsubstituted C₁₋₃₀ heteroalkylene, substituted or unsubstituted C₃₋₃₀ cycloalkylene, substituted or unsubstituted C₃₋₃₀ heterocycloalkylene, substituted or unsubstituted C₆₋₃₀ arylene, substituted or unsubstituted C₃₋₃₀ heteroarylene, —O—, —C(O))—, —C(O))O—, —S—, —S(O)₂—, —N(R^{9a})—, or —C(O))N(R^{9b})—, wherein R^{9a} and R^{9b} may be each independently hydrogen, substituted or unsubstituted C₁₋₂₀ alkyl, substituted or unsubstituted C₃₋₂₀ cycloalkyl, or substituted or unsubstituted C₃₋₂₀ heterocycloalkyl.

It is to be understood that when L⁶ is a single bond, the moiety —R²³ is directly connected to the oxygen atom adjacent to the carbonyl group (i.e., —C(O))O—R²³).

In Formula (9), R²³ is a substituted or unsubstituted C₄₋₂₀ lactone-containing group or a substituted or unsubstituted C₄₋₂₀ sultone-containing group. The C₄₋₂₀ lactone-containing group and the C₄₋₂₀ sultone-containing group may be monocyclic, polycyclic, or fused polycyclic.

Exemplary monomers of Formula (9) may include one or more of the following:

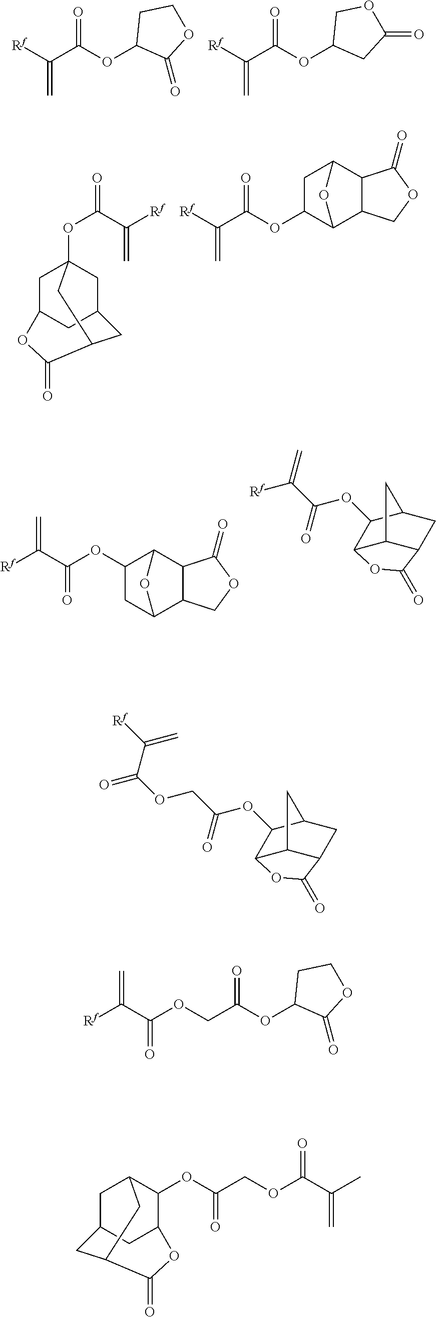

wherein R^{f} is as defined for Formula (9).

The polymer may include a repeating unit that is base-soluble and/or having a pKa of less than or equal to 12. For example, the repeating unit including a polar group pendant to the backbone of the polymer may be derived from one or more monomers of Formulae (10), (11), or (12):

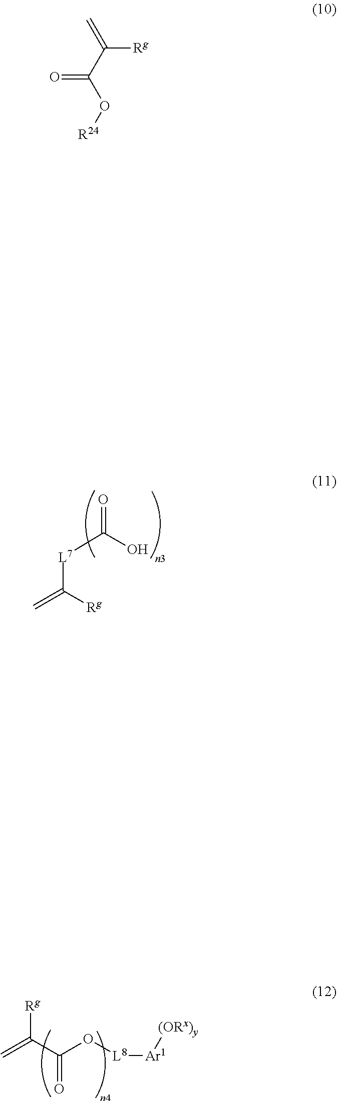

wherein each R^{g} may be hydrogen, fluorine, cyano, or substituted or unsubstituted C₁₋₁₀ alkyl. Preferably, R^{g} may be hydrogen, fluorine, or substituted or unsubstituted C₁₋₅ alkyl, typically methyl.

In Formula (10), R²⁴ may be substituted or unsubstituted C₁₋₁₀₀ or C₁₋₂₀ alkyl, typically C₁₋₁₂ alkyl; substituted or unsubstituted C₃₋₃₀ or C₃₋₂₀ cycloalkyl; or substituted or unsubstituted poly(C₁₋₃ alkylene oxide). Preferably, the substituted C₁₋₁₀₀ or C₁₋₂₀ alkyl, the substituted C₃₋₃₀ or C₃₋₂₀ cycloalkyl, and the substituted poly(C₁₋₃ alkylene oxide) are substituted with one or more of halogen, a fluoroalkyl group such as a C₁₋₄ fluoroalkyl group, typically fluoromethyl, a sulfonamide group —NH—S(O)₂—Y¹ where Y¹ is F or C₁₋₄ perfluoroalkyl (e.g., —NHSO₂CF₃), or a fluoroalcohol group (e.g., —C(CF₃)₂OH).

In Formula (11), L⁷ represents a single bond or a multivalent linking group chosen, for example, from optionally substituted aliphatic, such as C₁₋₆ alkylene or C₃₋₂₀ cycloalkylene, and aromatic hydrocarbons, and combinations thereof, optionally with one or more linking moieties chosen from —O—, —C(O)—, —C(O))O—, —S—, —S(O)₂—, —NR¹⁰²—, or —C(O))N(R¹⁰²)—, wherein R¹⁰² is chosen from hydrogen and optionally substituted C₁₋₁₀ alkyl. For example, the polymer may further include a repeating unit derived from one or more monomers of Formula (10) wherein L⁷ is a single bond or a multivalent linking group selected from substituted or unsubstituted C₁₋₂₀ alkylene, typically C₁₋₆ alkylene; substituted or unsubstituted C₃₋₂₀ cycloalkylene; typically, C₃₋₁₀ cycloalkylene; and substituted or unsubstituted C₆₋₂₄ arylene.

In Formula (11), n3 is an integer from 1 to 5, typically 1. It is to be understood that when n3 is 1, the group L⁷ is a divalent linking group. It is to be understood that when n3 is 2, the group L⁷ is a trivalent linking group. Similarly, it is to be understood that when n3 is 3, the group L⁷ is a tetravalent linking group; when n3 is 4, the group L⁷ is a pentavalent linking group; and when n3 is 5, the group L⁷ is a hexavalent linking group. Accordingly, in the context of Formula (10), the term “multivalent linking group” refers to any of a divalent, trivalent, tetravalent, pentavalent, and/or hexavalent linking groups. In some aspects, when n is 2 or greater, the carboxylic acid groups (—C(O))OH) may be connected to the same atom of the linking group L⁷. In other aspects, when n is 2 or greater, the carboxylic acid groups (—C(O)OH) may be connected to different atoms of the linking group L⁷.

In Formula (12), L⁸ represents a single bond or a divalent linking group. Preferably, L⁸ may be a single bond, substituted or unsubstituted C₆₋₃₀ arylene, or substituted or unsubstituted C₆₋₃₀ cycloalkylene.

In Formula (12), n4 is 0 or 1. It is to be understood that when n4 is 0, the moiety represented by —OC(O)—is a single bond such that L⁸ is directly connected to the alkenyl (vinylic) carbon atom.

In Formula (12), Ar¹ is a substituted C₅₋₆₀ aromatic group that optionally includes one or more aromatic ring heteroatoms chosen from N, O, S, or a combination thereof, wherein the aromatic group may be monocyclic, non-fused polycyclic, or fused polycyclic. When the C₅₋₆₀ aromatic group is polycyclic, the ring or ring groups may be fused (such as naphthyl or the like), non-fused, or a combination thereof. When the polycyclic C₅₋₆₀ aromatic group is non-fused, the ring or ring groups may be directly linked (such as biaryls, biphenyl, or the like) or may be bridged by a heteroatom (such as triphenylamino or diphenylene ether). In some aspects, the polycyclic C₅₋₆₀ aromatic group may include a combination of fused rings and directly linked rings (such as binaphthyl or the like).

In Formula (12), y may be an integer from 1 to 12, preferably from 1 to 6, and typically from 1 to 3. Each R^{x} may independently be hydrogen or methyl.

Non-limiting examples of monomers of Formulae (10), (11), or (12) include one or more of the following:

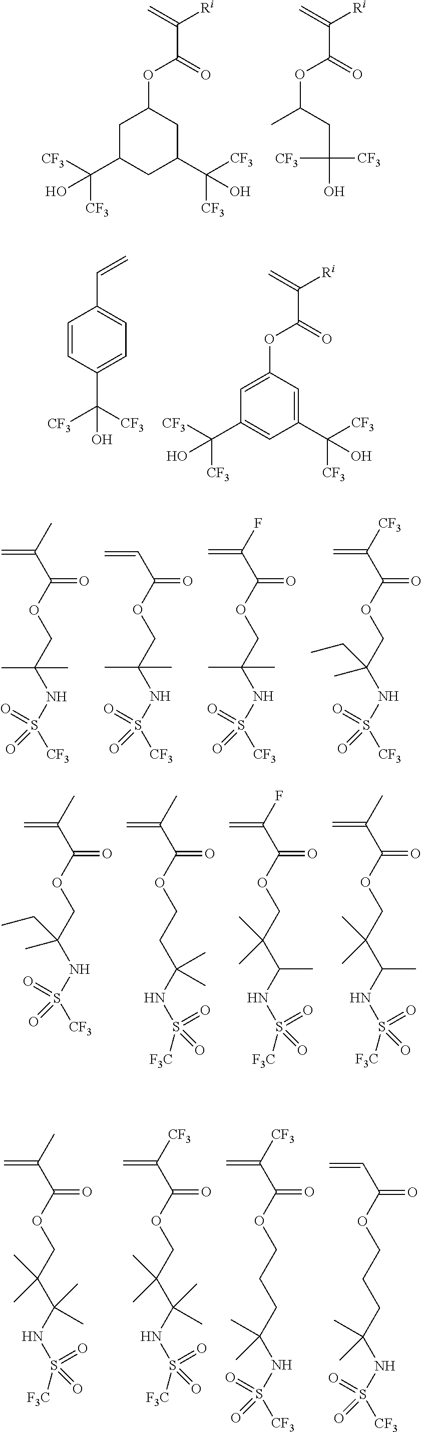

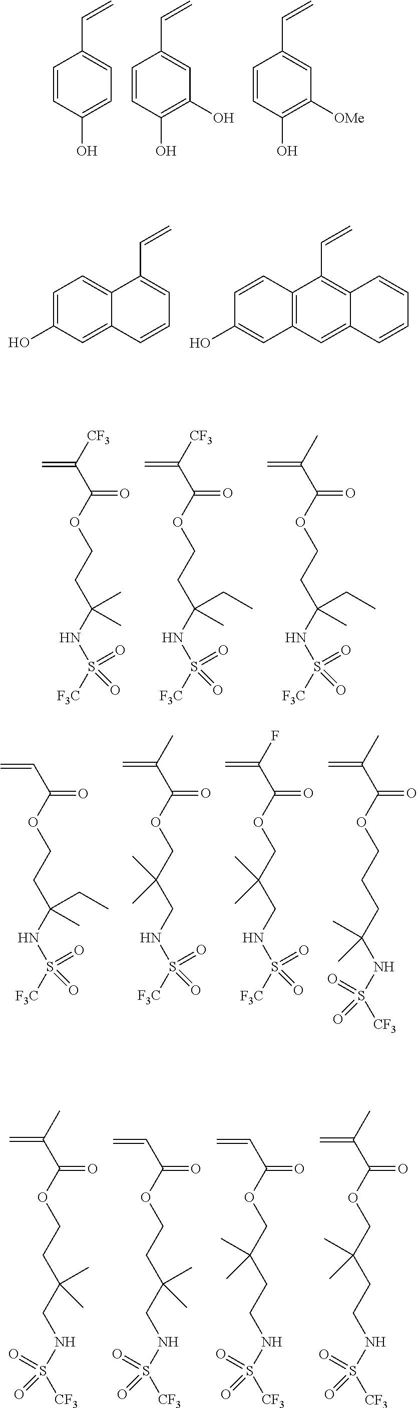

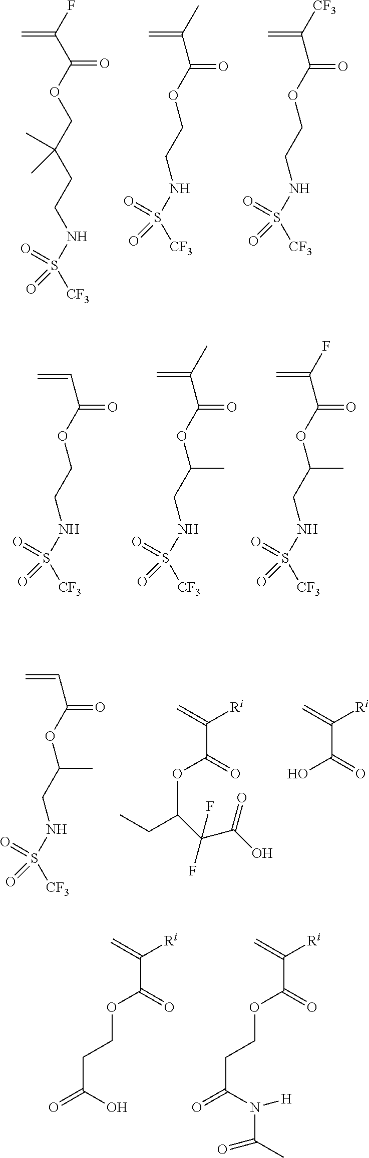

wherein Y¹ is as described above, and R′ is as defined for W in Formulae (10)-(12).

When present, the polymer typically comprises a repeating unit comprising a polar group (pendant to a backbone of the polymer) in an amount from 1 to 60 mol %, typically from 5 to 50 mol %, more typically from 5 to 40 mol %, based on total repeating units in the polymer.

Non-limiting exemplary polymers of the present invention include one or more of the following:

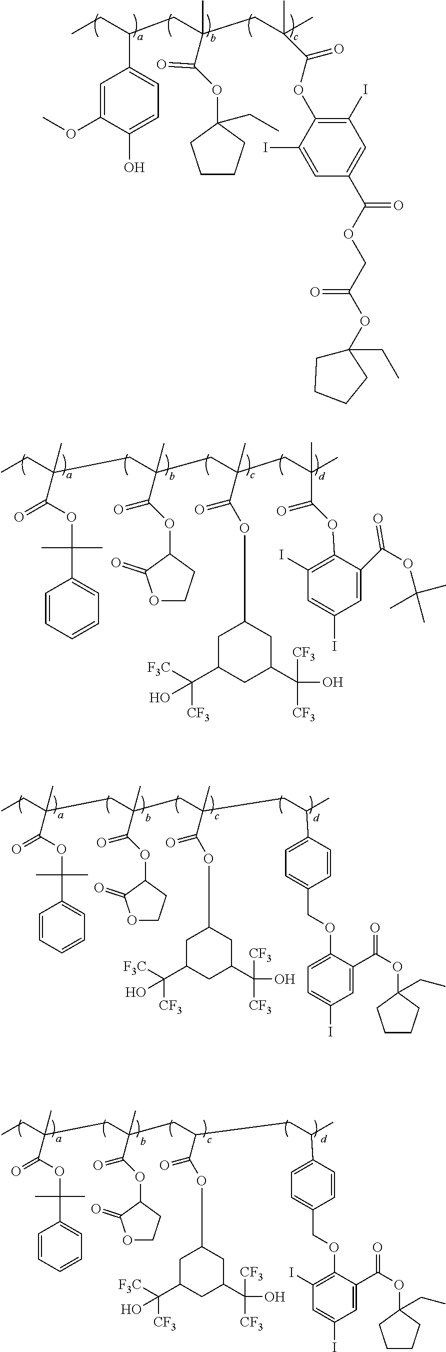

wherein a, b, and c, or a, b, c, and d represent the mole fractions for the respective repeating units of the polymer.

The polymer typically has a weight average molecular weight (M_{w}) from 1,000 to 50,000 Dalton (Da), preferably from 2,000 to 30,000 Da, more preferably 4,000 to 25,000 Da, and still more preferably from 5,000 to 25,000 Da. The polydispersity index (PDI) of the first polymer, which is the ratio of M_{w} to number average molecular weight (Mₙ) is typically from 1.1 to 3, and more typically from 1.1 to 2. Molecular weight values are determined by gel permeation chromatography (GPC) using polystyrene standards.

The polymer may be prepared using any suitable method(s) in the art. For example, one or more monomers corresponding to the repeating units described herein may be combined, or fed separately, using suitable solvent(s) and initiator, and polymerized in a reactor. For example, the polymer may be obtained by polymerization of the respective monomers under any suitable conditions, such as by heating at an effective temperature, irradiation with actinic radiation at an effective wavelength, or a combination thereof.

Also provided are photoresist compositions including the inventive polymer, a photoacid generator (PAG), and a solvent.

Suitable PAGs can generate an acid that, during post-exposure bake (PEB), causes cleavage of acid-labile groups present on a polymer of the photoresist composition. The PAG may be in non-polymeric form or in polymeric form, for example, present in a polymerized repeating unit of the polymer as described above, or as part of a different polymer. In some embodiments, the PAG may be included in the composition as a non-polymerized PAG compound, as a repeating unit of a polymer having a PAG moiety that is derived from a polymerizable PAG monomer, or as a combination thereof.

Suitable non-polymeric PAG compounds may have Formula G⁺ A⁻, wherein G⁺ is an organic cation chosen from iodonium cations substituted with two alkyl groups, two aryl groups, or a combination of alkyl and aryl groups; and sulfonium cations substituted with three alkyl groups, three aryl groups, or a combination of alkyl and aryl groups, and A⁻ is a non-polymerizable organic anion. Particularly suitable non-polymeric organic anions include those, the conjugated acids of which have a pKa of from —15 to 1. Particularly preferred anions are fluorinated alkyl sulfonates and fluorinated sulfonimides.

Useful non-polymeric PAG compounds are known in the art of chemically amplified photoresists and include, for example: onium salts, for example, triphenylsulfonium trifluoromethanesulfonate, (p-tert-butoxyphenyl)diphenylsulfonium trifluoromethanesulfonate, tris(p-tert-butoxyphenyl)sulfonium trifluoromethanesulfonate, triphenylsulfonium p-toluenesulfonate; di-t-butyphenyliodonium perfluorobutanesulfonate, and di-t-butyphenyliodonium camphorsulfonate. Non-ionic sulfonates and sulfonyl compounds are also known to function as photoacid generators, e.g., nitrobenzyl derivatives, for example, 2-nitrobenzyl-p-toluenesulfonate, 2,6-dinitrobenzyl-p-toluenesulfonate, and 2,4-dinitrobenzyl-p-toluenesulfonate; sulfonic acid esters, for example, 1,2,3-tris(methanesulfonyloxy)benzene, 1,2,3-tris(trifluoromethanesulfonyloxy)benzene, and 1,2,3-tris(p-toluenesulfonyloxy)benzene; diazomethane derivatives, for example, bis(benzenesulfonyl)diazomethane, bis(p-toluenesulfonyl)diazomethane; glyoxime derivatives, for example, bis-O-(p-toluenesulfonyl)-α-dimethylglyoxime, and bis-O-(n-butanesulfonyl)-a-dimethylglyoxime; sulfonic acid ester derivatives of an N-hydroxyimide compound, for example, N-hydroxysuccinimide methanesulfonic acid ester, N-hydroxysuccinimide trifluoromethanesulfonic acid ester; and halogen-containing triazine compounds, for example, 2-(4-methoxyphenyl)-4,6-bis(trichloromethyl)-1,3,5-triazine, and 2-(4-methoxynaphthyl)-4,6-bis(trichloromethyl)-1,3,5-triazine. Suitable non-polymerized photoacid generators are further described in U.S. Pat. No. 8,431,325 to Hashimoto et al. in column 37, lines 11-47 and columns 41-91. Other suitable sulfonate PAGs include sulfonated esters and sulfonyloxy ketones, nitrobenzyl esters, s-triazine derivatives, benzoin tosylate, t-butylphenyl a-(p-toluenesulfonyloxy)acetate, and t-butyl α-(p-toluenesulfonyloxy)acetate; as described in U.S. Pat. Nos. 4,189,323 and 8,431,325.

Typically, when the photoresist composition includes a non-polymeric photoacid generator, it is present in the photoresist composition in an amount of from 0.3 to 65 wt %, more typically 1 to 20 wt %, based on total solids of the photoresist composition.

In some embodiments, G⁺ may be a sulfonium cation of Formula (13) or an iodonium cation of Formula (14):

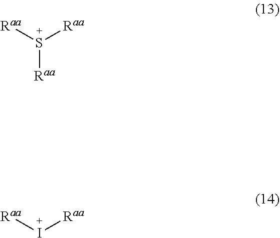

In Formulae (13) and (14), each R^{aa} is independently substituted or unsubstituted C₁₋₂₀ alkyl, substituted or unsubstituted C₃₋₂₀ cycloalkyl, substituted or unsubstituted C₂₋₂₀ alkenyl, substituted or unsubstituted C₆₋₃₀ aryl, substituted or unsubstituted C₃₋₃₀ heteroaryl, substituted or unsubstituted C₇₋₂₀ arylalkyl, or substituted or unsubstituted C₄₋₂₀ heteroarylalkyl. Each R^{aa} may be either separate or connected to another group R^{aa} via a single bond or a divalent linking group to form a ring. Each R^{aa} optionally may include as part of its structure a divalent linking group. Each R^{aa} independently may optionally comprise an acid-labile group chosen, for example, from tertiary alkyl ester groups, secondary or tertiary aryl ester groups, secondary or tertiary ester groups having a combination of alkyl and aryl groups, tertiary alkoxy groups, acetal groups, or ketal groups.

Exemplary sulfonium cations of Formula (13) may include one or more of the following:

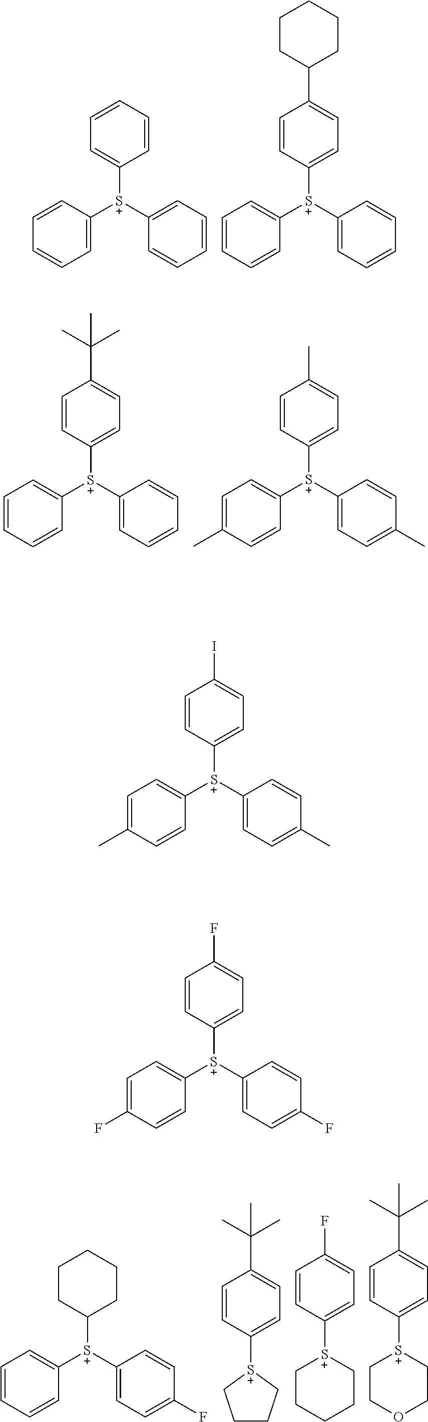

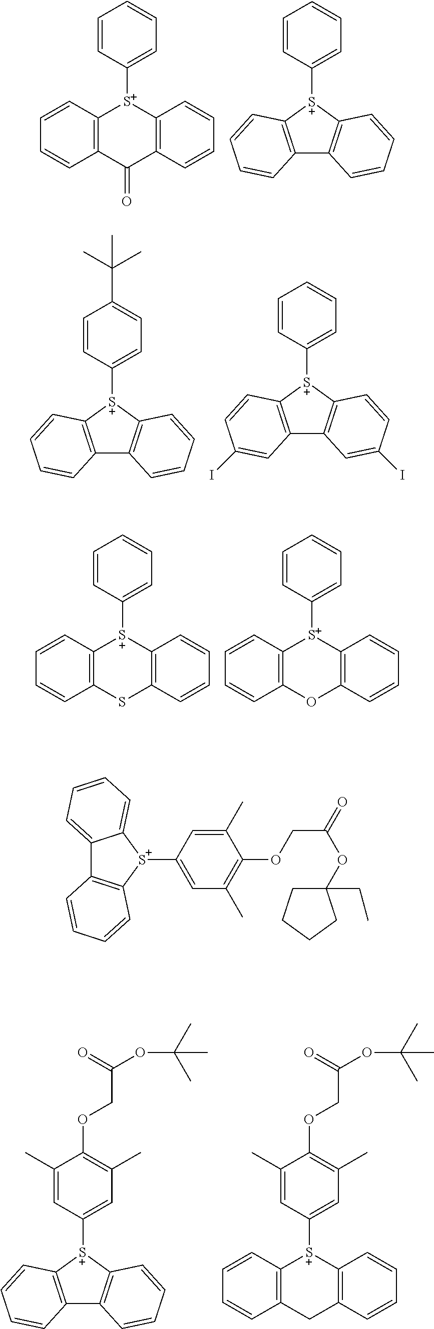

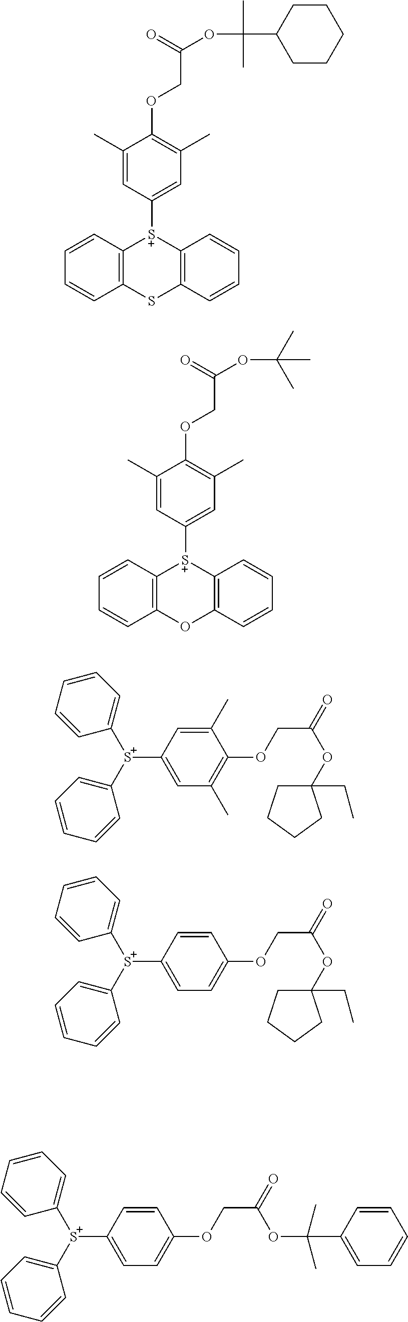 [SMILES: Cc1cc([S+]2c3ccccc3Sc3ccccc32)cc(C)c1OCC(=O)OC(C)(C)C1CCCCC1 ; Cc1cc([S+]2c3ccccc3Oc3ccccc32)cc(C)c1OCC(=O)OC(C)(C)C ; CCC1(OC(=O)COc2c(C)cc([S+](c3ccccc3)c3ccccc3)cc2C)CCCC1 ; CCC1(OC(=O)COc2ccc([S+](c3ccccc3)c3ccccc3)cc2)CCCC1 ; CC(C)(OC(=O)COc1ccc([S+](c2ccccc2)c2ccccc2)cc1)c1ccccc1]

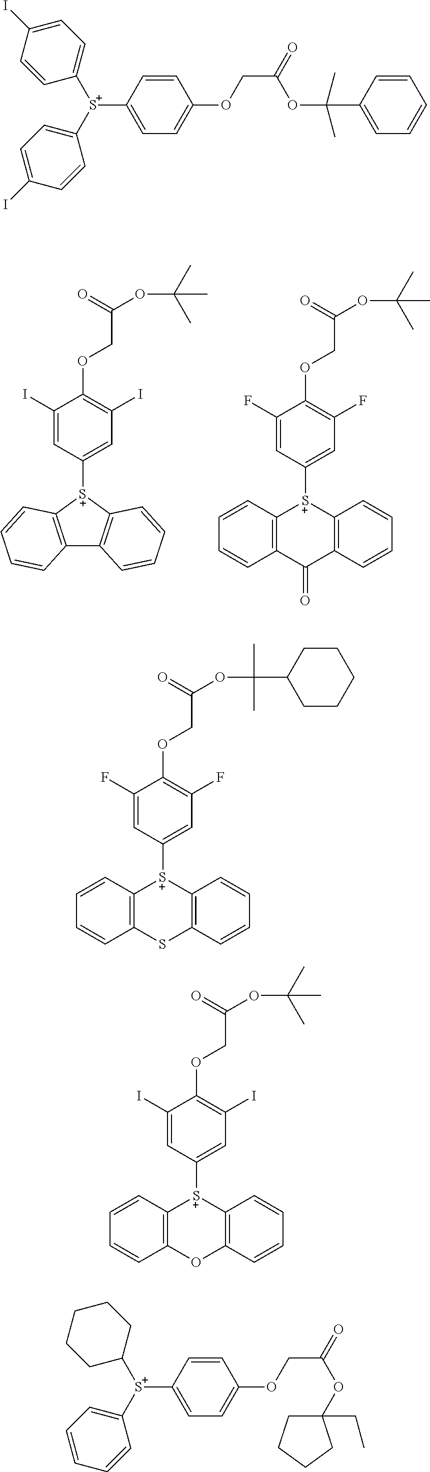 [SMILES: CC(C)(OC(=O)COc1ccc([S+](c2ccc(I)cc2)c2ccc(I)cc2)cc1)c1ccccc1 ; CC(C)(C)OC(=O)COc1c(I)cc(-[s+]2c3ccccc3c3ccccc32)cc1I ; CC(C)(C)OC(=O)COc1c(F)cc(-[s+]2c3ccccc3c(=O)c3ccccc32)cc1F ; CC(C)(OC(=O)COc1c(F)cc([S+]2c3ccccc3Sc3ccccc32)cc1F)C1CCCCC1 ; CC(C)(C)OC(=O)COc1c(I)cc([S+]2c3ccccc3Oc3ccccc32)cc1I ; CCC1(OC(=O)COc2ccc([S+](c3ccccc3)C3CCCCC3)cc2)CCCC1]

Exemplary iodonium cations of Formula (14) may include one or more of the following:

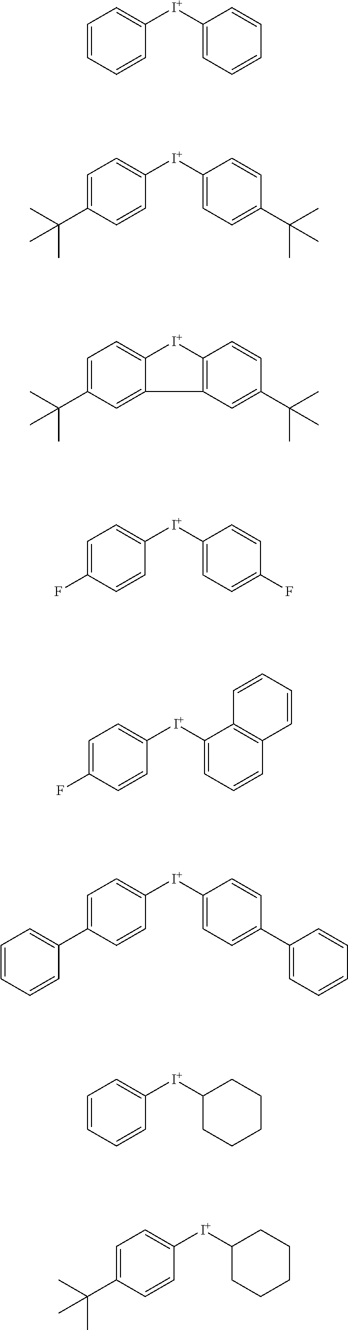

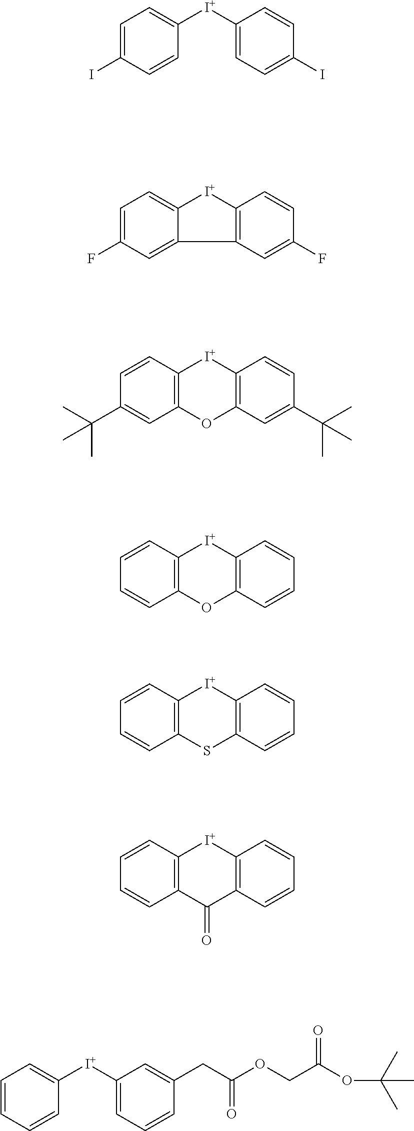

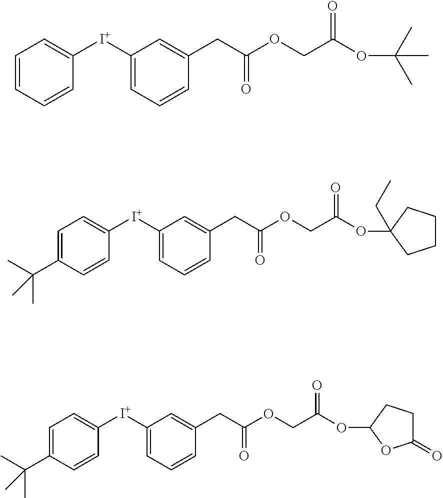

PAGs that are onium salts typically comprise an organic anion having a sulfonate group or a non-sulfonate-type group, such as sulfonamidate, sulfonimidate, methide, or borate.

Exemplary organic anions having a sulfonate group include one or more of the following:

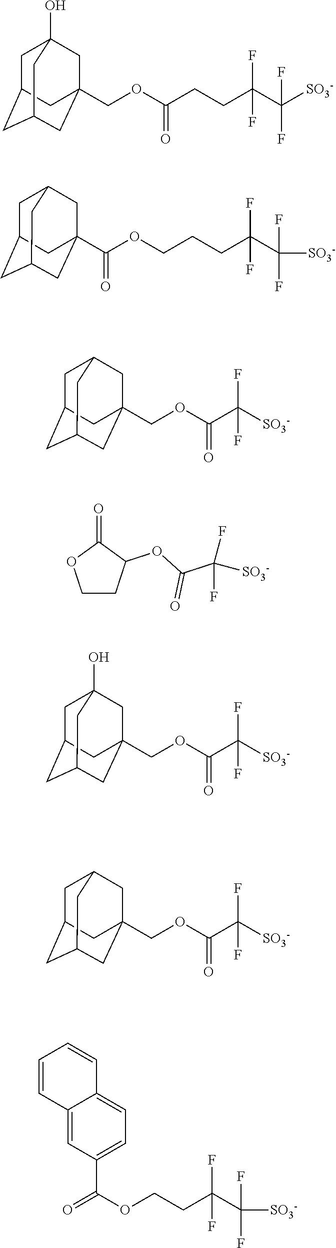

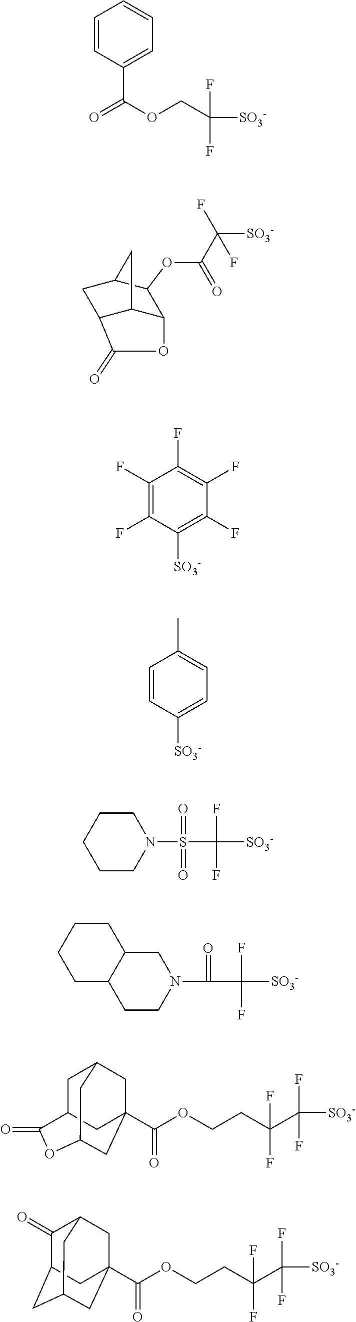

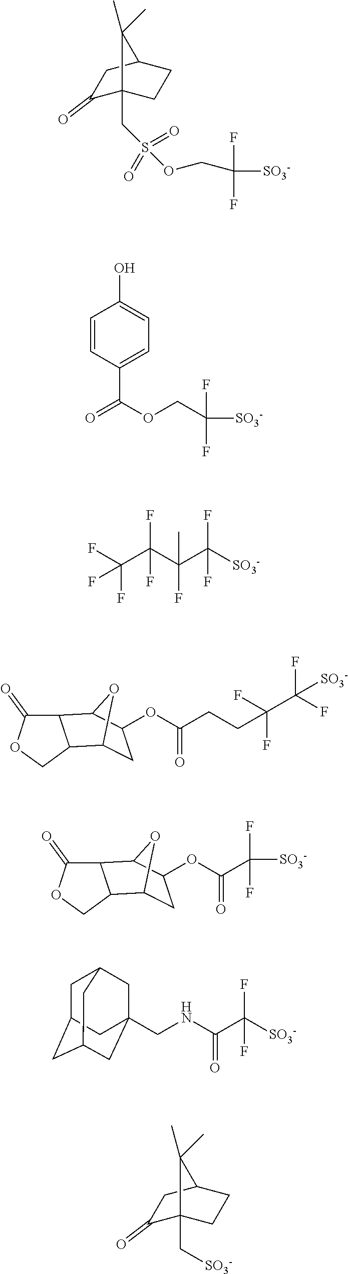

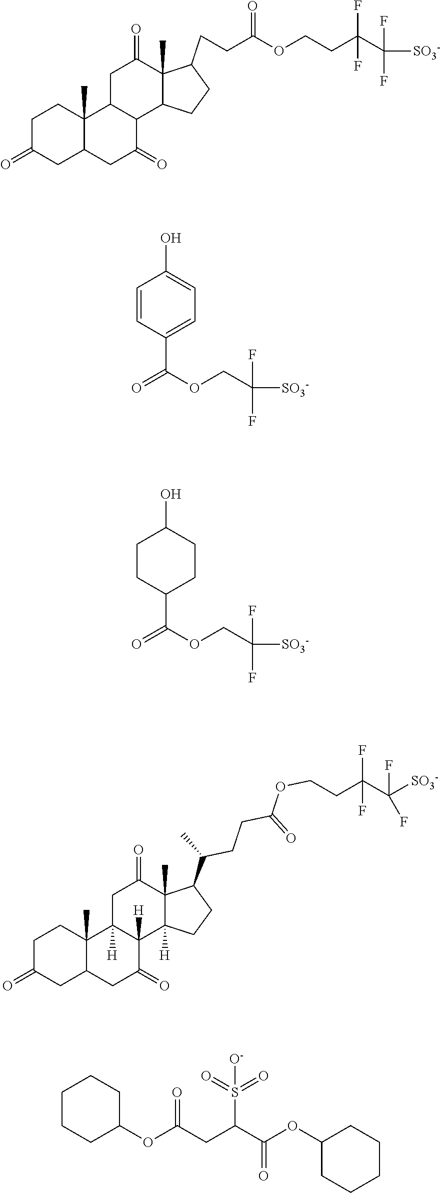

Exemplary non-sulfonated anions include one or more of the following:

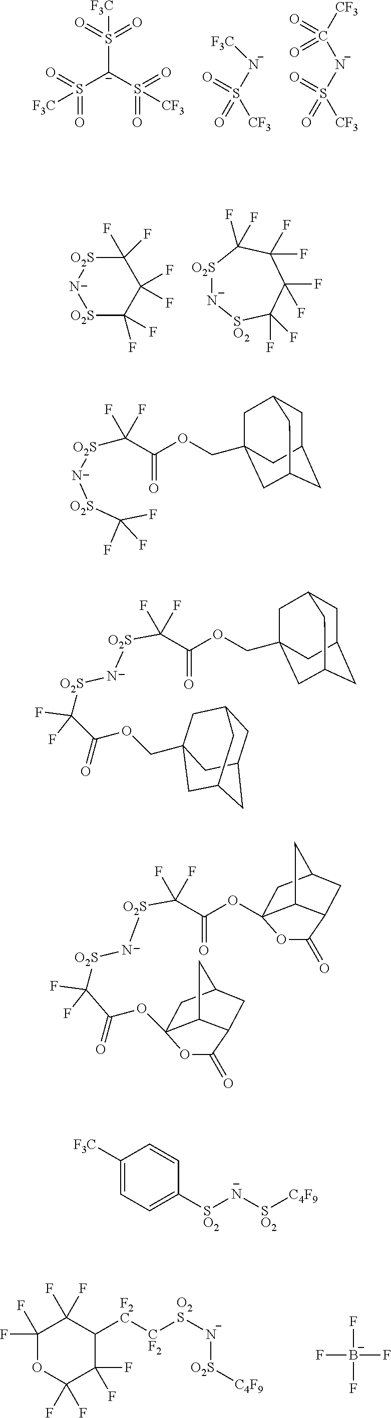

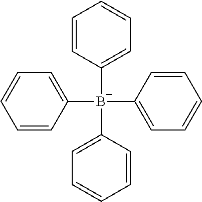 [SMILES: c1ccc([B-](c2ccccc2)(c2ccccc2)c2ccccc2)cc1]

The photoresist composition may optionally comprise a plurality of PAGs. The plurality of PAGs may be polymeric, non-polymeric, or may include both polymeric and non-polymeric PAGs. Preferably, each PAG of the plurality of PAGs is non-polymeric.

In one or more aspects, the photoresist composition may include a first photoacid generator that includes a sulfonate group on the anion, and the photoresist composition may include a second photoacid generator that is non-polymeric, wherein the second photoacid generator may include an anion that is free of sulfonate groups.

In some aspects, the polymer optionally may further include a repeating unit that comprises a PAG-containing moiety, for example a repeating unit derived from one or more monomers of Formula (15):

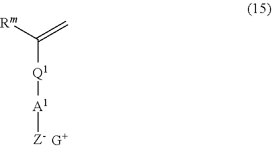

wherein R^{m} may be hydrogen, fluorine, cyano, or substituted or unsubstituted C₁₋₁₀ alkyl. Preferably, R^{m} is hydrogen, fluorine, or substituted or unsubstituted C₁₋₅ alkyl, typically methyl.

In Formula (15), Q¹ may be a single bond or a divalent linking group. Preferably, Q¹ may include 1 to 10 carbon atoms and at least one heteroatom, more preferably —C(O)—O—. A¹ may be one or more of substituted or unsubstituted C₁₋₃₀ alkylene, substituted or unsubstituted C₃₋₃₀ cycloalkylene, substituted or unsubstituted C₃₋₃₀ heterocycloalkylene, substituted or unsubstituted C₆₋₃₀ arylene, or substituted or unsubstituted C₃₋₃₀ heteroarylene. Preferably, A^{l} may be a divalent C₁₋₃₀ perfluoroalkylene group that is optionally substituted. Z⁻ is an anionic moiety, the conjugated acid of which typically has a pKa from −15 to 1. For example, Z⁻ may be a sulfonate, a carboxylate, an anion of a sulfonamide, an anion of a sulfonimide, or a methide anion. Particularly preferred anion moieties are fluorinated alkyl sulfonates and fluorinated sulfonimides. G⁺ is an organic cation as defined above. In some embodiments, G⁺ is an iodonium cation substituted with two alkyl groups, two aryl groups, or a combination of alkyl and aryl groups; or a sulfonium cation substituted with three alkyl groups, three aryl groups, or a combination of alkyl and aryl groups.

Exemplary monomers of Formula (15) may include one or more of the following:

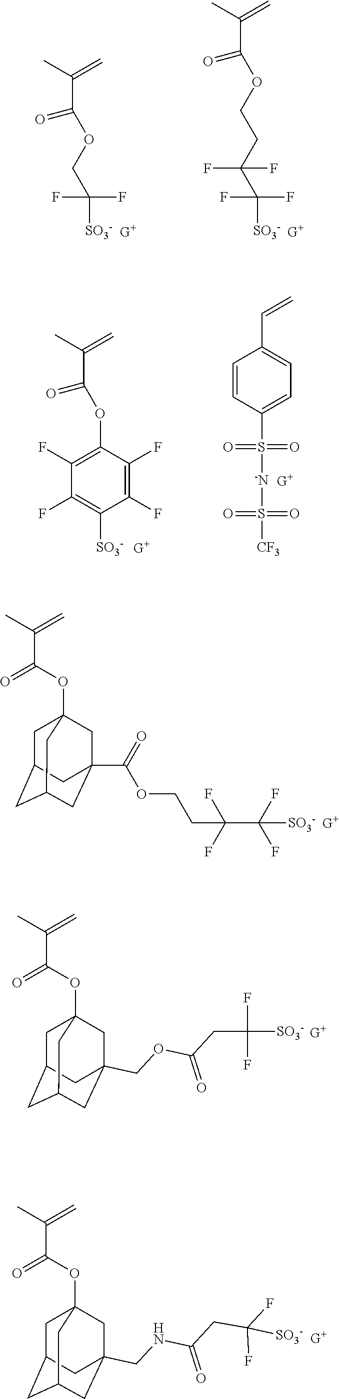

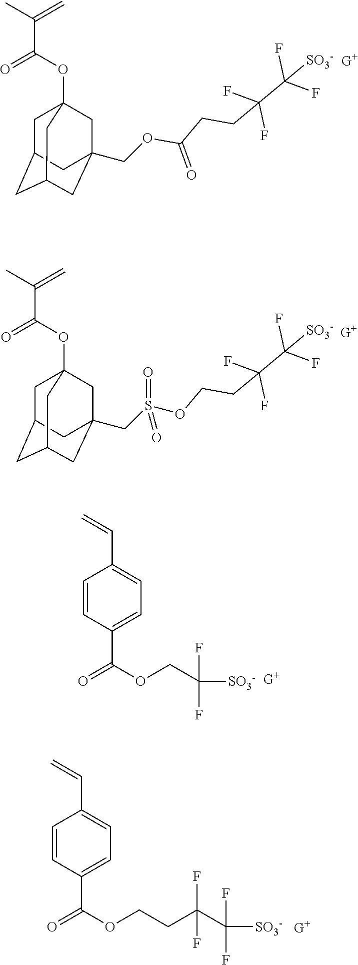

wherein G⁺ is an organic cation as defined herein.

When used, the repeating unit comprising a PAG moiety can be included in a polymer in an amount from 1 to 15 mol %, typically from 1 to 8 mol %, more typically from 2 to 6 mol %, based on total repeating units in the polymer.

The photoresist composition further includes a solvent for dissolving the components of the composition and to facilitate its coating on a substrate. Preferably, the solvent is an organic solvent conventionally used in the manufacture of electronic devices. Suitable solvents include, for example: aliphatic hydrocarbons such as hexane and heptane; aromatic hydrocarbons such as toluene and xylene; halogenated hydrocarbons such as dichloromethane, 1,2-dichloroethane and 1-chlorohexane; alcohols such as methanol, ethanol, 1-propanol, iso-propanol, tert-butanol, 2-methyl-2-butanol, 4-methyl-2-pentanol, and diacetone alcohol (4-hydroxy-4-methyl-2-pentanone); propylene glycol monomethyl ether (PGME); ethers such as diethyl ether, tetrahydrofuran, 1,4-dioxane, and anisole; ketones such as acetone, methyl ethyl ketone, methyl iso-butyl ketone, 2-heptanone, and cyclohexanone (CHO); esters such as ethyl acetate, n-butyl acetate, propylene glycol monomethyl ether acetate (PGMEA), ethyl lactate (EL), hydroxyisobutyrate methyl ester (HBM), and ethyl acetoacetate; lactones such as gamma-butyrolactone (GBL) and epsilon-caprolactone; lactams such as N-methyl pyrrolidone; nitriles such as acetonitrile and propionitrile; cyclic or non-cyclic carbonate esters such as propylene carbonate, dimethyl carbonate, ethylene carbonate, propylene carbonate, diphenyl carbonate, and propylene carbonate; polar aprotic solvents such as dimethyl sulfoxide and dimethyl formamide; water; and combinations thereof. Of these, preferred solvents are PGME, PGMEA, EL, GBL, HBM, CHO, and combinations thereof.

The total solvent content (i.e., cumulative solvent content for all solvents) in the photoresist compositions is typically from 40 to 99 wt %, for example, from 60 to 99 wt %, or from 85 to 99 wt %, based on total solids of the photoresist composition. The desired solvent content will depend, for example, on the desired thickness of the coated photoresist layer and coating conditions.

The polymer is typically present in the photoresist composition in an amount from 10 to 99.9 wt %, typically from 25 to 99 wt %, and more typically from 40 to 95 wt %, based on total solids of the photoresist composition. It will be understood that “total solids” includes the polymer, PAGs, the additive, and other non-solvent components.

In some aspects, the photoresist composition may further include a material that comprises one or more base-labile groups (a “base-labile material”). As referred to herein, base-labile groups are functional groups that can undergo cleavage reaction to provide polar groups such as hydroxyl, carboxylic acid, sulfonic acid, and the like, in the presence of an aqueous alkaline developer after exposure and post-exposure baking steps. The base-labile group will not react significantly (e.g., will not undergo a bond-breaking reaction) prior to a development step of the photoresist composition that comprises the base-labile group. Thus, for instance, a base-labile group will be substantially inert during pre-exposure soft-bake, exposure, and post-exposure bake steps. By “substantially inert” it is meant that ≤5%, typically ≤1%, of the base-labile groups (or moieties) will decompose, cleave, or react during the pre-exposure soft-bake, exposure, and post-exposure bake steps. The base-labile group is reactive under typical photoresist development conditions using, for example, an aqueous alkaline photoresist developer such as a 0.26 normal (N) aqueous solution of tetramethylammonium hydroxide (TMAH). For example, a 0.26 N aqueous solution of TMAH may be used for single puddle development or dynamic development, e.g., where the 0.26 N TMAH developer is dispensed onto an imaged photoresist layer for a suitable time such as 10 to 120 seconds (s). An exemplary base-labile group is an ester group, typically a fluorinated ester group. Preferably, the base-labile material is substantially not miscible with and has a lower surface energy than the polymer and other solid components of the photoresist composition. When coated on a substrate, the base-labile material can thereby segregate from other solid components of the photoresist composition to a top surface of the formed photoresist layer.

In some aspects, the base-labile material may be a polymeric material, also referred to herein as a base-labile polymer, which may include one or more repeating units comprising one or more base-labile groups. For example, the base-labile polymer may comprise a repeating unit comprising 2 or more base-labile groups that are the same or different. A preferred base-labile polymer includes at least one repeating unit comprising 2 or more base-labile groups, for example a repeating unit comprising 2 or 3 base-labile groups.

The base-labile polymer may be a polymer comprising a repeating unit derived from one or more monomers of Formula (16):

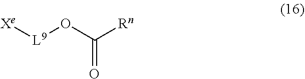

wherein X^{e} is a polymerizable group selected from C₂ alkenyl and (meth)acrylic, L⁹ is a divalent linking group; and Rⁿ is substituted or unsubstituted C₁₋₂₀ fluoroalkyl, provided that the carbon atom bonded to the carbonyl (—C(O))—) in Formula (16) is substituted with at least one fluorine atom. Exemplary monomers of Formula (16) may include one or more of the following:

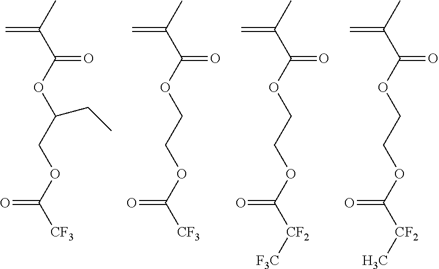

The base-labile polymer may include a repeating unit including two or more base-labile groups. For example, the base-labile polymer can include a repeating unit derived from one or more monomers of Formula (17):

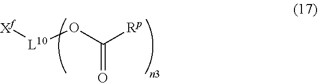

wherein X^{f} and R^{p} are as defined in Formula (16) for X^{e} and Rⁿ, respectively; L¹⁰ is a polyvalent linking group including one or more of substituted or unsubstituted C₁₋₂₀ alkylene, substituted or unsubstituted C₃₋₂₀ cycloalkylene, —C(O))—, or —C(O))O—; and n3 may be an integer of 2 or greater, for example 2 or 3. Exemplary monomers of Formula (17) may include one or more of the following:

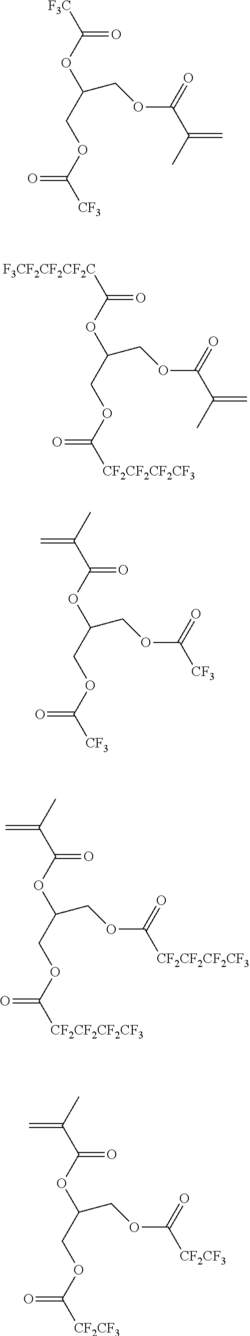

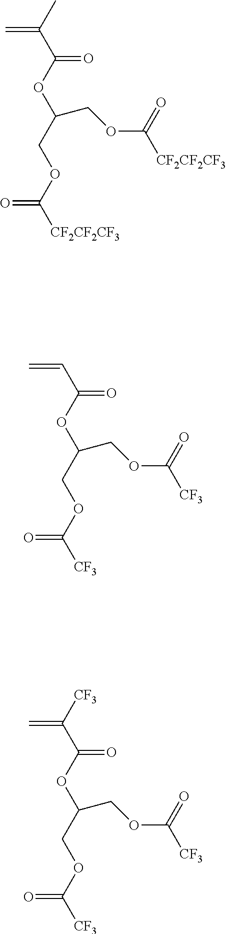

The base-labile polymer may include a repeating unit including one or more base-labile groups. For example, the base-labile polymer can include a repeating unit derived from one or more monomers of Formula (18):

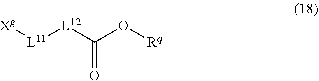

wherein X^{g} and R^{q} are as defined in Formula (16) for X^{e} and Rⁿ, respectively; L¹¹ is a divalent linking group; and L¹² is substituted or unsubstituted C₁₋₂₀ fluoroalkylene wherein the carbon atom bonded to the carbonyl (—C(O))—) in Formula (18) is substituted with at least one fluorine atom. Exemplary monomers of Formula (18) may include one or more of the following:

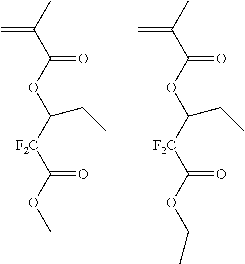 [SMILES: C=C(C)C(=O)OC(CC)C(F)(F)C(=O)OC ; C=C(C)C(=O)OC(CC)C(F)(F)C(=O)OCC]

In some aspects, a base-labile polymer may comprise one or more base-labile groups and one or more acid-labile groups, such as one or more acid-labile ester moieties (e.g., t-butyl ester) or acid-labile acetal groups. For example, the base-labile polymer may comprise a repeating unit including a base-labile group and an acid-labile group, i.e., wherein both a base-labile group and an acid-labile group are present on the same repeating unit. In another example, the base-labile polymer may comprise a first repeating unit comprising a base-labile group and a second repeating unit comprising an acid-labile group. Preferred photoresists of the invention can exhibit reduced defects associated with a resist relief image formed from the photoresist composition.

The base-labile polymer may be prepared using any suitable methods in the art, including those described herein for the first and second polymers. For example, the base-labile polymer may be obtained by polymerization of the respective monomers under any suitable conditions, such as by heating at an effective temperature, irradiation with actinic radiation at an effective wavelength, or a combination thereof. Additionally, or alternatively, one or more base-labile groups may be grafted onto the backbone of a polymer using suitable methods.

In some aspects, the base-labile material is a single molecule comprising one more base-labile ester groups, preferably one or more fluorinated ester groups. The base-labile materials that are single molecules typically have a M_{w} in the range from 50 to 1,500 Da. Exemplary base-labile materials include one or more of the following:

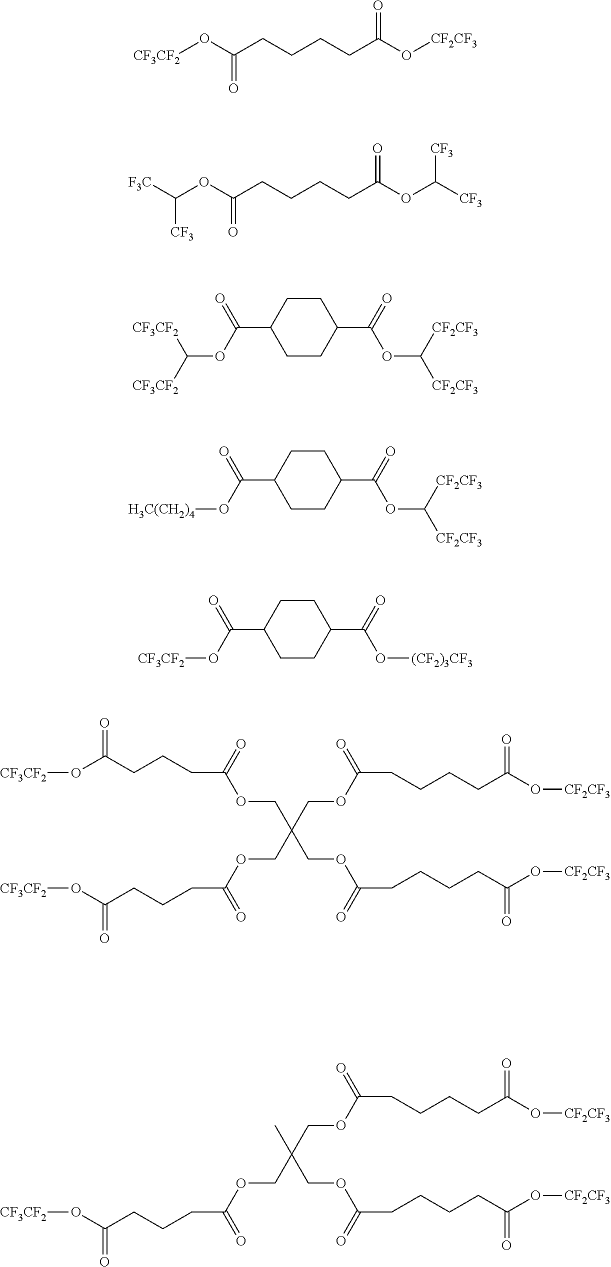

When present, the base-labile material is typically present in the photoresist compositions in an amount of from 0.01 to 10 wt %, typically 1 to 5 wt %, based on total solids of the photoresist composition.

Additionally, or alternatively, to the base-labile polymer, the photoresist compositions may further include one or more polymers in addition to and different from the photoresist polymer described above. For example, the photoresist compositions may include an additional polymer as described above but different in composition, or a polymer that is similar to those described above but does not include each of the requisite repeating units. Additionally, or alternatively, the one or more additional polymers may include those well known in the photoresist art, for example, those chosen from polyacrylates, polyvinylethers, polyesters, polynorbornenes, polyacetals, polyethylene glycols, polyamides, polyacrylamides, polyphenols, novolacs, styrenic polymers, polyvinyl alcohols, or combinations thereof.

The photoresist composition may further include one or more additional, optional additives. For example, optional additives may include actinic and contrast dyes, anti-striation agents, plasticizers, speed enhancers, sensitizers, photo-decomposable quenchers (PDQ) (and, also known as photo-decomposable bases), basic quenchers, thermal acid generators, surfactants, and the like, or combinations thereof. If present, the optional additives are typically present in the photoresist compositions in an amount of from 0.01 to 10 wt %, based on total solids of the photoresist composition.

PDQs generate a weak acid upon irradiation. The acid generated from a photo-decomposable quencher is not strong enough to react rapidly with acid-labile groups that are present in the resist matrix. Exemplary photo-decomposable quenchers include, for example, photo-decomposable cations, and preferably those also useful for preparing strong acid generator compounds, paired with an anion of a weak acid (pKa>1) such as, for example, an anion of a C₁₋₂₀ carboxylic acid or C₁₋₂₀ sulfonic acid. Exemplary carboxylic acids include formic acid, acetic acid, propionic acid, tartaric acid, succinic acid, cyclohexanecarboxylic acid, benzoic acid, salicylic acid, and the like. Exemplary sulfonic acids include p-toluene sulfonic acid, camphor sulfonic acid and the like. In a preferred embodiment, the photo-decomposable quencher is a photo-decomposable organic zwitterion compound such as diphenyliodonium-2-carboxylate.

The photo-decomposable quencher may be in non-polymeric or polymer-bound form. When in polymeric form, the photo-decomposable quencher is present in polymerized units on the first polymer or second polymer. The polymerized units containing the photo-decomposable quencher are typically present in an amount from 0.1 to 30 mole %, preferably from 1 to 10 mole % and more preferably from 1 to 2 mole %, based on total repeating units of the polymer.

Exemplary basic quenchers include, for example, linear aliphatic amines such as tributylamine, trioctylamine, triisopropanolamine, tetrakis(2-hydroxypropyl)ethylenediamine:n-tert-butyldiethanolamine, tris(2-acetoxy-ethyl) amine, 2,2′,2″,2″-(ethane-1,2-diylbis(azanetriyl))tetraethanol, 2-(dibutylamino)ethanol, and 2,2′,2″-nitrilotriethanol; cyclic aliphatic amines such as 1-(tert-butoxycarbonyl)-4-hydroxypiperidine, tert-butyl 1-pyrrolidinecarboxylate, tert-butyl 2-ethyl-1H-imidazole-1-carboxylate, di-tert-butyl piperazine-1,4-dicarboxylate, and N-(2-acetoxy-ethyl)morpholine; aromatic amines such as pyridine, di-tert-butyl pyridine, and pyridinium; linear and cyclic amides and derivatives thereof such as N,N-bis(2-hydroxyethyl)pivalamide, N,N-diethylacetamide, N¹,N¹,N³,N³-tetrabutylmalonamide, 1-methylazepan-2-one, 1-allylazepan-2-one, and tert-butyl 1,3-dihydroxy-2-(hydroxymethyl)propan-2-ylcarbamate; ammonium salts such as quaternary ammonium salts of sulfonates, sulfamates, carboxylates, and phosphonates; imines such as primary and secondary aldimines and ketimines; diazines such as optionally substituted pyrazine, piperazine, and phenazine; diazoles such as optionally substituted pyrazole, thiadiazole, and imidazole; and optionally substituted pyrrolidones such as 2-pyrrolidone and cyclohexyl pyrrolidine.

The basic quenchers may be in non-polymeric or polymer-bound form. When in polymeric form, the quencher may be present in repeating units of the polymer. The repeating units containing the quencher are typically present in an amount of from 0.1 to 30 mole %, preferably from 1 to 10 mole % and more preferably from 1 to 2 mole %, based on total repeating units of the polymer.

Exemplary surfactants include fluorinated and non-fluorinated surfactants and can be ionic or non-ionic, with non-ionic surfactants being preferable. Exemplary fluorinated non-ionic surfactants include perfluoro C4 surfactants such as FC-4430 and FC-4432 surfactants, available from 3M Corporation; and fluorodiols such as POLYFOX PF-636, PF-6320, PF-656, and PF-6520 fluorosurfactants from Omnova. In an aspect, the photoresist composition further includes a surfactant polymer including a fluorine-containing repeating unit.

Patterning methods using the photoresist compositions of the invention will now be described. Suitable substrates on which the photoresist compositions can be coated include electronic device substrates. A wide variety of electronic device substrates may be used in the present invention, such as: semiconductor wafers; polycrystalline silicon substrates; packaging substrates such as multichip modules; flat panel display substrates; substrates for light emitting diodes (LEDs) including organic light emitting diodes (OLEDs); and the like, with semiconductor wafers being typical. Such substrates are typically composed of one or more of silicon, polysilicon, silicon oxide, silicon nitride, silicon oxynitride, silicon germanium, gallium arsenide, aluminum, sapphire, tungsten, titanium, titanium-tungsten, nickel, copper, and gold. Suitable substrates may be in the form of wafers such as those used in the manufacture of integrated circuits, optical sensors, flat panel displays, integrated optical circuits, and LEDs. Such substrates may be any suitable size. Typical wafer substrate diameters are 200 to 300 millimeters (mm), although wafers having smaller and larger diameters may be suitably employed according to the present invention. The substrates may include one or more layers or structures which may optionally include active or operable portions of devices being formed.

Typically, one or more lithographic layers such as a hardmask layer, for example, a spin-on-carbon (SOC), amorphous carbon, or metal hardmask layer, a CVD layer such as a silicon nitride (SiN), a silicon oxide (SiO), or silicon oxynitride (SiON) layer, an organic or inorganic underlayer, or combinations thereof, are provided on an upper surface of the substrate prior to coating a photoresist composition of the present invention. Such layers, together with an overcoated photoresist layer, form a lithographic material stack.

Optionally, a layer of an adhesion promoter may be applied to the substrate surface prior to coating the photoresist compositions. If an adhesion promoter is desired, any suitable adhesion promoter for polymer films may be used, such as silanes, typically organosilanes such as trimethoxyvinylsilane, triethoxyvinylsilane, hexamethyldisilazane, or an aminosilane coupler such as gamma-aminopropyltriethoxysilane. Particularly suitable adhesion promoters include those sold under the AP 3000, AP 8000, and AP 9000S designations, available from DuPont Electronics & Imaging (Marlborough, Massachusetts).

The photoresist composition may be coated on the substrate by any suitable method, including spin coating, spray coating, dip coating, doctor blading, or the like. For example, applying the layer of photoresist may be accomplished by spin coating the photoresist in solvent using a coating track, in which the photoresist is dispensed on a spinning wafer. During dispensing, the wafer is typically spun at a speed of up to 4,000 rotations per minute (rpm), for example, from 200 to 3,000 rpm, for example, 1,000 to 2,500 rpm, for a period from 15 to 120 seconds to obtain a layer of the photoresist composition on the substrate. It will be appreciated by those skilled in the art that the thickness of the coated layer may be adjusted by changing the spin speed and/or the total solids of the composition. A photoresist composition layer formed from the compositions of the invention typically has a dried layer thickness from 3 to 30 micrometers (um), preferably from greater than 5 to 30 um, and more preferably from 6 to 25 μm.

The photoresist composition is typically next soft-baked to minimize the solvent content in the layer, thereby forming a tack-free coating and improving adhesion of the layer to the substrate. The soft bake is performed, for example, on a hotplate or in an oven, with a hotplate being typical. The soft bake temperature and time will depend, for example, on the photoresist composition and thickness. The soft bake temperature is typically from 80 to 170° C., and more typically from 90 to 150° C. The soft bake time is typically from 10 seconds to 20 minutes, more typically from 1 minute to 10 minutes, and still more typically from 1 minute to 2 minutes. The heating time can be readily determined by one of ordinary skill in the art based on the ingredients of the composition.

The photoresist layer is next pattern-wise exposed to activating radiation to create a difference in solubility between exposed and unexposed regions. Reference herein to exposing a photoresist composition to radiation that is activating for the composition indicates that the radiation can form a latent image in the photoresist composition. The exposure is typically conducted through a patterned photomask that has optically transparent and optically opaque regions corresponding to regions of the resist layer to be exposed and unexposed, respectively. Such exposure may, alternatively, be conducted without a photomask in a direct writing method, typically used for e-beam lithography. The activating radiation typically has a wavelength of sub-400 nm, sub-300 nm or sub-200 nm, with 248 nm (KrF), 193 nm (ArF), 13.5 nm (EUV) wavelengths ore-beam lithography being preferred. Preferably, the activating radiation is 248 nm radiation. The methods find use in immersion or dry (non-immersion) lithography techniques. The exposure energy is typically from 1 to 200 millijoules per square centimeter (mJ/cm²), preferably from 10 to 100 mJ/cm² and more preferably from 20 to 50 mJ/cm², dependent upon the exposure tool and components of the photoresist composition.

Following exposure of the photoresist layer, a post-exposure bake (PEB) of the exposed photoresist layer is performed. The PEB can be conducted, for example, on a hotplate or in an oven, with a hotplate being typical. Conditions for the PEB will depend, for example, on the photoresist composition and layer thickness. The PEB is typically conducted at a temperature from 70 to 150° C., preferably from 75 to 120° C., and a time from 30 to 120 seconds. A latent image defined by the polarity-switched (exposed regions) and unswitched regions (unexposed regions) is formed in the photoresist.

The exposed photoresist layer is then developed with a suitable developer to selectively remove those regions of the layer that are soluble in the developer while the remaining insoluble regions form the resulting photoresist pattern relief image. In the case of a positive-tone development (PTD) process, the exposed regions of the photoresist layer are removed during development and unexposed regions remain. Conversely, in a negative-tone development (NTD) process, the exposed regions of the photoresist layer remain, and unexposed regions are removed during development. Application of the developer may be accomplished by any suitable method such as described above with respect to application of the photoresist composition, with spin coating being typical. The development time is for a period effective to remove the soluble regions of the photoresist, with a time of from 5 to 60 seconds being typical. Development is typically conducted at room temperature.

Suitable developers for a PTD process include aqueous base developers, for example, quaternary ammonium hydroxide solutions such as tetramethylammonium hydroxide (TMAH), preferably 0.26 normal (N) TMAH, tetraethylammonium hydroxide, tetrabutylammonium hydroxide, sodium hydroxide, potassium hydroxide, sodium carbonate, potassium carbonate, and the like. Suitable developers for an NTD process are organic solvent-based, meaning the cumulative content of organic solvents in the developer is 50 wt % or more, typically 95 wt % or more, 98 wt % or more, or 100 wt %, based on total weight of the developer. Suitable organic solvents for the NTD developer include, for example, those chosen from ketones, esters, ethers, hydrocarbons, and mixtures thereof The developer is typically 2-heptanone or n-butyl acetate.

A coated substrate may be formed from the photoresist compositions of the invention. Such a coated substrate includes: (a) a substrate having one or more layers to be patterned on a surface thereof; and (b) a layer of the photoresist composition over the one or more layers to be patterned.

The photoresist pattern may be used, for example, as an etch mask, thereby allowing the pattern to be transferred to one or more sequentially underlying layers by known etching techniques, typically by dry-etching such as reactive ion etching The photoresist pattern may, for example, be used for pattern transfer to an underlying hardmask layer which, in turn, is used as an etch mask for pattern transfer to one or more layers below the hardmask layer. If the photoresist pattern is not consumed during pattern transfer, it may be removed from the substrate by known techniques, for example, oxygen plasma ashing. The photoresist compositions may, when used in one or more such patterning processes, be used to fabricate semiconductor devices such as memory devices, processor chips (CPUs), graphics chips, optoelectronic chips, LEDs, OLEDs, as well as other electronic devices.

The invention is further illustrated by the following non-limiting examples.

### EXAMPLES

### Example 1

The synthetic scheme for the monomer designated MD2 is shown in Scheme 1.

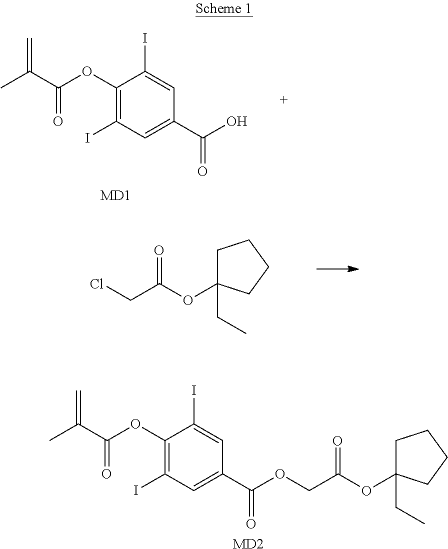

To a mixture of 3,5-diiodo-4-(methacryloyloxy)benzoic acid (MD1, 20 grams (g), 43.66 millimoles (mmol)), and 1-ethylcyclopentyl 2-chloroacetate (13 g, 68.18 mmol) in 150 milliliters (mL) of N,N-dimethylformamide (DMF) under nitrogen atmosphere was added cesium carbonate (15 g, 77.75 mmol) in a single portion. The reaction mixture was stirred at room temperature for 24 hours. The resulting mixture was filtered to remove insoluble inorganics and the filtrate was poured into 200 mL of deionized (DI) water to produce the crude product as an oily residue. The crude product was purified by filtration through a short plug of silica-gel using heptane/ethyl acetate in volume ratio of 10:1 as eluent. To the combined fractions of the pure product was added 100 mg of the inhibitor dibutylhydroxytoluene (BHT), and the solvent was removed under reduced pressure to produce 13.0 g (49%) of the monomer MD2 as a colorless solid. ¹H NMR (Acetone-d6), δ: 8.52 (s, 2H, 2ArH), 6.49 (s, 1H, CH═CH), 6.06 (s, 1H, CH═CH), 4.86 (s, 2H, CH₂), 2.15 (m, 4H, 2CH₂), 2.08 (m, 4H, 2CH₂) 1.63 (m, 2H, CH₂), 0.88 (t, 3H, CH).

### Example 2

The synthetic scheme for the monomer designated MD3 is shown in Scheme 2.

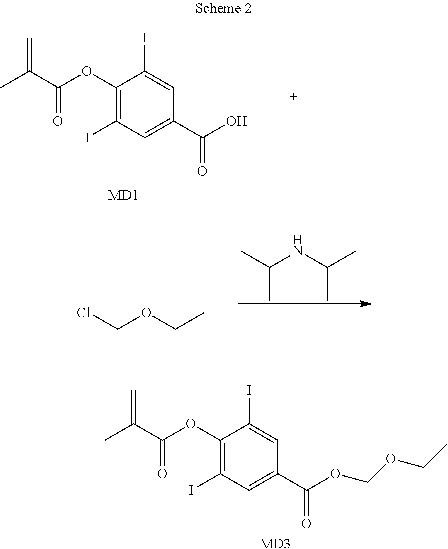

To a mixture of 3,5-diiodo-4-(methacryloyloxy)benzoic acid (5 g, 10.91 mmol) in 50 mL of tetrahydrofuran (THF) was added chloromethylethyl ether (1.0 g, 10.57 mmol). Diiosopropylamine (1.11 g, 10.96 mmol) was then added thereto slowly and the mixture was stirred overnight at room temperature. The resulting ammonium slat was removed by filtration and the THF was removed under reduced pressure. The resulting residue was dissolved in 50 mL dichloromethane (DCM) and washed with a 0.1 molar (M) aqueous solution of ammonium chloride (50 mL). The DCM solution was then filtered through a short pad of silica gel. The solvent was removed under reduced pressure to produce the product MD3 as a white solid. Yield 3.3 g. ¹H NMR (Acetone-d6), δ: 8.51 (s, 2H, ArH), 6.51 (s, 1H, CH═CH), 5.87 (s, 1H, CH═CH), 5.60 (s, 2H, CH₂), 3.79 (t, 2H, CH₂), 2.17 (s, 3h, CH₃) 1.23 (q, 2H, CH₃).

### Example 3

The synthetic scheme for the monomer designated MD4 is shown in Scheme 3.

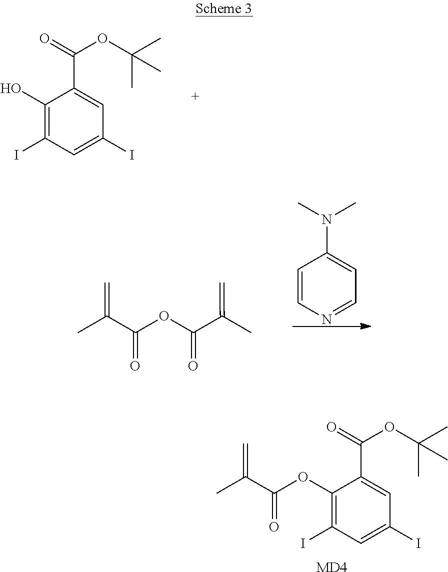

To a solution of tert-butyl 2-hydroxy-3,5-diiodobenzoate (TBDISA) (16.0 g, 35.67 mmol) in 150 mL of THF was added a solution of N,N-dimethylaminopyridine in 5 mL of DCM. To the mixture was added methacrylic anhydride (6.47 g, 41.88 mmol). The content was stirred at room temperature for 24 hours, and then the reaction mixture was concentrated to yield a pale-yellow oil (23.7 g). The crude product was dissolved in 30 mL of DCM and purified by filtration through a short plug of silica-gel. To the combined fractions of the pure product was added 100 mg of the inhibitor dibutylhydroxytoluene (BHT), and the solvent was removed under reduced pressure to produce 16.0 g (86.7%) of the monomer MD4 as pale-yellow liquid. ^{1H} NMR (Acetone-d6) δ: 8.45 (s, 1H, ArH), 8.25 (s, 1H, ArH), 6.48 (s, 1H, CH═CH), 5.90 (s, 1H, CH═CH), 2.50 (s, 3H, CH₃),1.50 (s, 9H, C(CH₃)₃).

### Example 4

The synthetic scheme for the monomer designated MD5 is shown in Scheme 4.

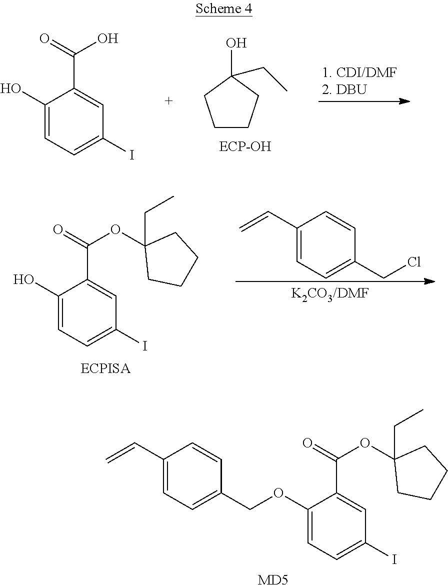

2-hydroxy—S—iodo-benzoic acid (30.7 g, 116.28 mmol) and carbonyldiimidazole (CDI, 28.28 g, 174.42 mmol) were dissolved in DMF (150 mL). The resulting solution was heated to 50° C., stirred for 2.5 h, and then cooled to room temperature. 1,8-diazabicyclo(5.4.0)udec-7-ene (DBU, 7.08 g, 151.16 mmol) and ECP-OH (17.26 g, 151.16 mmol) were then added and the reaction mixture was stirred at room temperature for 48 h. The reaction mixture was washed with heptane to extract the product and concentrated to yield 24.5 g of crude ECPISA which was used in the next step without further purification.

To a mixture of the crude ECPISA (15.0 g, 41.64 mmol) and 4-vinylbenzyl chloride (7.63 g, 49.97 mmol) in 150 mL of DMF under nitrogen atmosphere was added potassium carbonate (8.63 g, 62.47 mmol). The reaction mixture was stirred at room temperature for 24 hours. The mixture was diluted with 200 mL of ethyl acetate and washed twice with 250 mL of DI water. The aqueous layer was washed with an additional 200 mL of ethyl acetate. The combined organic layers were then washed twice with 400 mL of DI water and dried over sodium sulfate, filtered, and the solvent was removed under reduced pressure to produce the crude product as a pale-yellow liquid. The crude product was purified by filtration through a short plug of silica-gel using heptane/ethyl acetate in volume ratio of 10:1 as eluent. To the combined fractions of the pure product was added 100 mg of BHT, and the solvent was removed under reduced pressure to yield 13 g (86.7%) of the monomer MD5 as colorless solid. ¹H NMR (Acetone-d6), δ: 7.81 (s, 1H, ArH), 7.73 (d, 2H, ArH), 7.46 (d, 2H, ArH), 7.39 (d, 2H, ArH), 7.01 (d, 1H, ArH), 6.74 (d, 1H, —CH═CH), 5.83 (d, 1H, CH═CH), 5.25 (d, 1H, CH═CH), 5.23 (s, 2H, CH₂), 2.08 (m, 2H, CH₂), 1.95 (m, 2H, CH₂) 1.63-1.44 (m, 6H, 3CH₂), 0.80 (t, 3H, CH).

### Example 5

The synthetic scheme for the monomer designated MD6 is shown in Scheme 5.

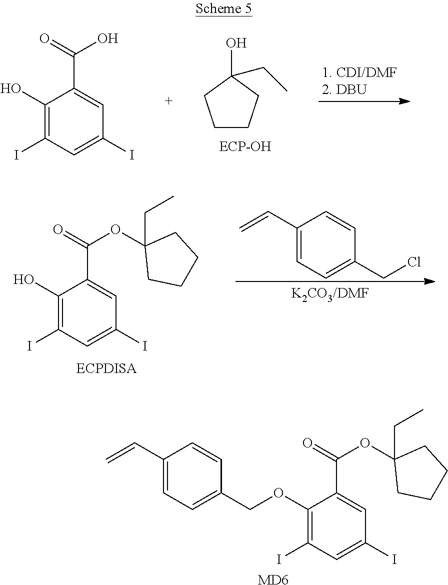

A mixture of 35-diiodosalicylic acid (25.0 g, 64 mmol) and carbonyldiimidazole (CDI, 15.59 g, 96.17 mmol) was dissolved in DMF (150 mL). The resulting solution was stirred under nitrogen atmosphere for 1 h. 1,8-diazabicyclo(5.4.0)udec-7-ene (DBU, 3.9 g, 25.65 mmol) and ECP-OH (9.52 g, 83.35 mmol) were then added and the reaction mixture was stirred at room temperature for 96 h. The reaction mixture was washed with heptane (4×150 mL) to extract the product and the combined extract solution was washed with DI water (2×250 mL). The organic layer was concentrated to yield 21.3 g of crude product ECPDISA which was used in the next step without further purification.

To a mixture of the crude ECPDISA (12.0 g, 24.69 mmol) and 4-vinylbenzyl chloride (4.52 g, 29.62 mmol) in 150 mL of DMF under nitrogen atmosphere was added potassium carbonate (5.12 g, 24.69 mmol) in a single portion. The reaction mixture was stirred at room temperature for 24 hours. The mixture was diluted with 200 mL of ethyl acetate and was washed twice with 250 mL of DI water. The aqueous layer was washed with an additional 200 mL of ethyl acetate. The combined organic layers were then washed twice with 250 mL of DI water, dried over sodium sulfate, filtered, and the solvent was removed under reduced pressure to produce a crude product as a liquid. The crude product was purified by filtration through a short plug of silica-gel using heptane containing 5 wt % of ethyl acetate as eluent. To the combined fractions of the pure product was added 100 mg of BHT, and the solvent was removed under reduced pressure to produce 11.2 g (75.3%) of the monomer MD5 as colorless solid. ¹H NMR (Acetone-d6) δ: 8.35 (s, 1H, ArH), 7.89 (s, 1H, ArH), 7.52-7.43 (m, 4H, 4ArH), 6.78 (d, 1H, —CH═CH), 5.88 (d, 1H, CH═CH), 5.29 (d, 1H, CH═CH), 4.95 (s, 2H, CH₂), 2.05 (m, 2H, CH₂), 1.99 (m, 2H, CH₂) 1.72-1.55 (m, 6H, 3CH₂), 0.83 (t, 3H, CH).

### Example 6

This example describes the synthesis of comparative polymers P1, P2, and P5 and of inventive polymers P3, P4, P6, P7, P8, and P9. The following monomers represent one or more of the structures that were used to prepare each of the comparative polymers and inventive polymers.

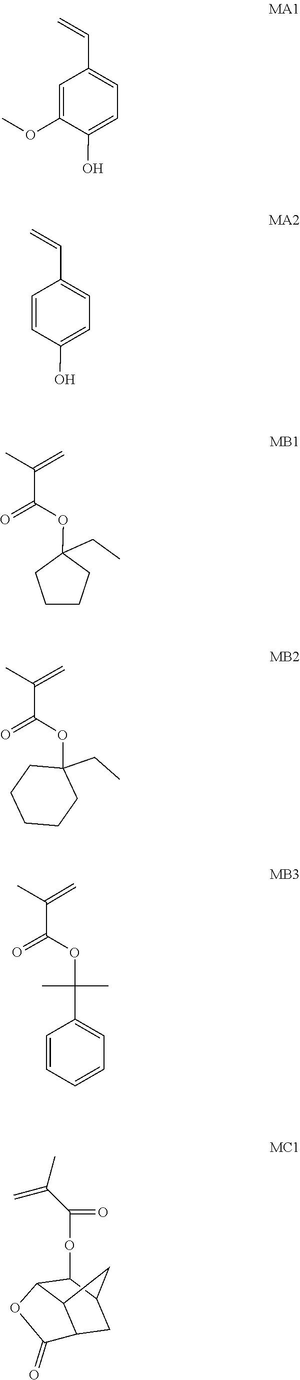

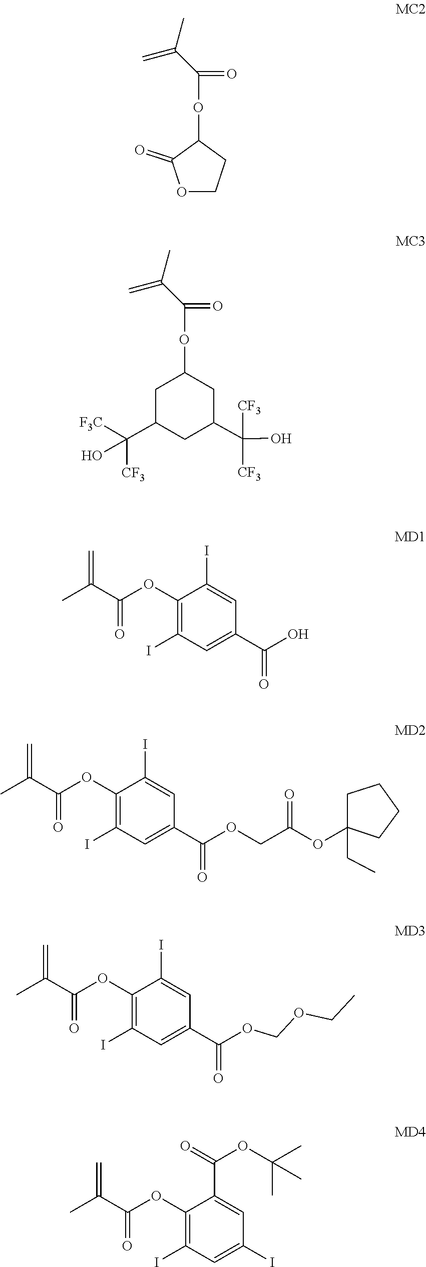

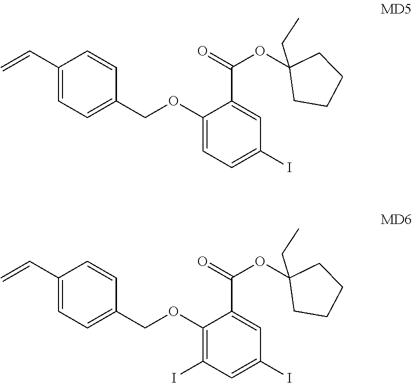 [SMILES: C=Cc1ccc(COc2ccc(I)cc2C(=O)OC2(CC)CCCC2)cc1 ; C=Cc1ccc(COc2c(I)cc(I)cc2C(=O)OC2(CC)CCCC2)cc1]

Comparative polymer P1 was prepared from the monomers MA1, MB1, and MC1, at a molar feed ratio of 50/40/10. A feed solution was made by dissolving MA1 (15.0 g, 100 mmol), MB1 (14.561 g, 80 mmol), and MC1 (4.44 g, 20 mmol) in 35 g of PGMEA. An initiator solution was separately prepared by dissolving 1.79 g of the azo initiator dimethyl 2,2′-azobis(2-methylpropionate) (obtained as V-601 from Wako Pure Chemical Industries, Ltd.) in 6.43 g of PGMEA/tetrahydrofuran (1:1 by wt %) mixture.

The polymerization was performed in a 3-neck round bottom flask fitted with a water condenser and a thermometer to monitor the reaction in the flask. The reactor was charged with 17.9 g of PGMEA and heated to 75° C. The feed solution and the initiator solution were each fed into the reactor using syringe pumps over 4 hours. The contents were then stirred for an additional 2 hours. The contents were subsequently cooled to room temperature, diluted with 20 g of THF, and precipitated into 800 mL of a 7:3 (w/w) mixture of heptane and isopropanol. The resulting copolymer P1 was isolated by filtration. A second precipitation was performed by dissolving the crude polymer in 50 g of THF and precipitation into 800 mL of a 7:3 (w/w) mixture of DI water/methanol.

Each of the polymers in Table 1 was prepared using a similar procedure to that described above for the preparation of Comparative polymer P1, except the monomers and molar feed ratios as specified in Table 1 were used.

TABLE 1

| | Monomer | Monomer | Monomer | Monomer |
|---|---|---|---|---|
| | 1 | 2 | 3 | 4 |
| Polymer | (mol %) | (mol %) | (mol %) | (mol %) |
| P1 (comparative) | MA1 (50) | MB1 (40) | MC1 (10) | |
| P2 (comparative) | MA1 (50) | MB1 (40) | MD1(10) | |
| P3 | MA1 (50) | MB1 (40) | MD2 (10) | |
| P4 | MA1 (60) | MB2 (40) | MD2 (10) | |
| P5 (comparative) | MB3 (45) | MC2 (45) | MC3 (10) | |
| P6 | MB3 (35) | MC2 (45) | MC3 (10) | MD4 (10) |
| P7 | MB3 (35) | MC2 (45) | MC3 (10) | MD2 (10) |
| P8 | MB3 (35) | MC2 (45) | MC3 (10) | MD5 (10) |
| P9 | MB3 (35) | MC2 (45) | MC3 (10) | MD6 (10) |

### Photoresist Compositions and Evaluation

Photoresist compositions were prepared by combining the components indicated in Table 2, where the amounts are expressed in weight percent (wt %) based on 100 wt % of total non-solvent component. The total solids content for the photoresist compositions was 3.3 wt %. The photoresist compositions were prepared in a solvent mixture of propylene glycol monomethyl ether acetate (PGMEA) and methyl-2-hydroxyisobutryate in a weight ratio of 1:1.

The resulting photoresist compositions were shaken on a mechanical shaker and then filtered through a PTFE disk-shaped filter having a 0.2 micron pore size. 200 mm silicon wafers overcoated with a BARC stack (60 nm-thickness AR3 antireflectant over an 80 nm-thickness AR40A antireflectant (DuPont Electronics & Industrial) were each spin-coated with a respective photoresist composition on a TEL Clean Track ACT 8 wafer track and softbaked at 110° C. for 60 seconds to provide a photoresist layer with a target thickness of about 100 nm. The resist layer thickness was measured with a THERMA-WAVE OP7350. The wafers were exposed with 248 nm radiation on a Canon FPA-5000 ES4 scanner at exposure doses between 3 and 53 millijoules per square centimeter (mJ/cm²). The wafers were subject to post-exposure bake (PEB) at 100° C. for 60 seconds, developed with MF-CD26 TMAH developer (DuPont Electronics & Imaging) for 60 seconds, rinsed with DI water, and dried. Photoresist layer thickness measurements were made in exposed and unexposed regions of the layer. A contrast curve for each wafer was generated by plotting the remaining photoresist layer thickness in the exposed regions versus dose. Dose-to-clear (E0) was determined from the contrast curve as the exposure dose at which the remaining photoresist layer thickness was less than 10% of the original coated thickness. Unexposed film thickness loss (UFTL) was determined based on the photoresist layer thickness measurements in the unexposed regions. The results are shown in Table 2.

TABLE 2

| Photoresist | | | | E₀ | UFTL |
|---|---|---|---|---|---|
| Composition | Polymer | PAG | Additive | (mJ/cm²) | (Å) |
| PR-1* | P1 | PAG-1 | Q1 | 12.8 | −32.0 |
| | (79.53) | (17.69) | (2.78) | | |
| PR-2* | P2 | PAG-1 | Q1 | 16.5 | −398.0 |
| | (79.53) | (17.69) | (2.78) | | |
| PR-3 | P3 | PAG-1 | Q1 | 17.9 | −6.0 |
| | (79.53) | (17.69) | (2.78) | | |
| PR-4 | P4 | PAG-1 | Q1 | 18.8 | −21.0 |
| | (79.53) | (17.69) | (2.78) | | |

*denotes a comparative photoresist composition

The structures of the PAG (PAG-1) and the additive (Q1) were as follows:

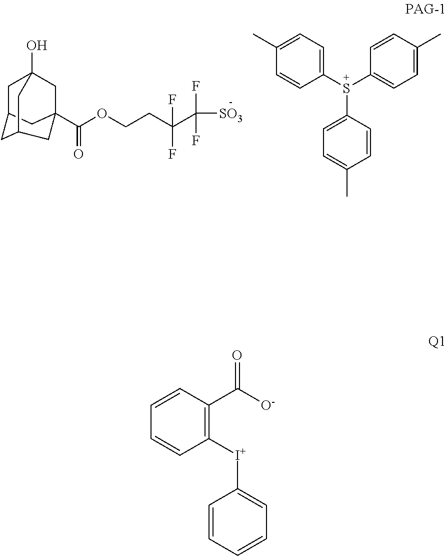

As shown above in Table 2, the photoresist compositions PR-3 and PR-4 including a polymer derived from the inventive compound achieved improved UFTL and an improved Eₒ (increased Eₒ) relative to the comparative photoresist compositions PR-1 and PR-2 that included the polymer that was not derived from the inventive compound.

The photoresist compositions PR-1, PR-2, PR-3 ,and PR-4 were evaluated for line/space patterning under KrF exposure. 200 mm silicon wafers overcoated with a BARC stack (60 nm-thickness AR3 antireflectant over 80 nm-thickness AR40A antireflectant (DuPont Electronics & Industrial) were each spin-coated with a respective photoresist composition on a TEL Clean Track ACT 8 wafer track and softbaked at 110° C. for 60 seconds to provide a photoresist layer with a thickness of about 90 nm. The wafers were each exposed with 248 nm radiation on a CANNON FPA-5000 ES4 scanner (NA=0.8, outer sigma=0.85, inner sigma=0.57) using a mask having 120 nm line/space (Us) patterns. The wafers were post-exposure baked at 100° C. for 60 seconds, developed with MF-CD26 TMAH developer (DuPont Electronics & Imaging) for 60 seconds, rinsed with DI water, and dried. Critical dimension (CD) measurements of the formed 1/s patterns were made with a Hitachi S-9380 CD SEM. Sizing energy (E_{size}), and line width roughness (LWR) were determined based on the CD measurements. Sizing energy is the irradiation energy at which the target 120 nm 1/s pattern was resolved.

The results are shown in Table 3.

TABLE 3

| Photoresist | E_{size} | LWR |
|---|---|---|
| Composition | (mJ/cm²) | (nm) |
| PR-1* | 61.7 | 7.35 |

| | |
|---|---|
| PR-2* | Printing failure |

| | | |
|---|---|---|
| PR-3 | 101.4 | 6.23 |
| PR-4 | 101.5 | 6.90 |

*denotes a comparative photoresist composition

As shown above in Table 3, the photoresist compositions PR-3 and PR-4 including a polymer derived from the inventive compound achieved improved LWR (i.e., a decreased LWR) and an improved E_{size} (increased E_{size}) relative to the photoresist compositions PR-1 and PR-2 that included a polymer that was not derived from the inventive compound.

Photoresist compositions were prepared by dissolving solid components in solvents using the materials and proportions set forth in Table 4 (specified in wt %, based on 100 wt % total solid components) to a total solids content of 3.5 wt %. The resulting mixtures were shaken on a mechanical shaker and then filtered through a PTFE disk-shaped filter having a 0.2 micron pore size. 200 mm silicon wafers overcoated with a BARC stack (60 nm-thickness AR3 antireflectant over 80 nm-thickness AR40A antireflectant (DuPont Electronics & Imaging) were each spin-coated with a respective photoresist composition on a TEL Clean Track ACT 8 wafer track and softbaked at 110° C. for 60 seconds to provide a photoresist layer with a thickness of 100 nm. The wafers were each exposed with 248 nm radiation on a CANON FPA-5000 ES4 scanner (NA=0.8, outer sigma=0.85, inner sigma=0.57) using a mask having 120 nm CD and 240 nm pitch trench patterns. The wafers were post-exposure baked at 100° C. for 60 seconds, developed with MF-CD26 TMAH developer (DuPont Electronics & Imaging) for 60 seconds, rinsed with DI water, and dried. Critical dimension (CD) measurements of the formed 1/s patterns were made with a Hitachi S-9380 CD SEM. Sizing energy (E_{size}), and line width roughness (LWR) based on the CD measurements. Sizing energy is the irradiation energy at which the target 120 nm pattern was resolved. The results are shown in Table 4.

TABLE 4

| Photoresist | | | | E_{size} | LWR |
|---|---|---|---|---|---|
| Composition | Polymer | PAG | Additive | (mJ/cm²) | (nm) |
| PR-5* | P5 | PAG-2 | Q1 | 122.44 | 6.57 |
| | (78.45) | (19.614) | (1.930) | | |
| PR-6 | P6 | PAG-2 | Q1 | 141.93 | 6.47 |
| | (78.45) | (19.614) | (1.930) | | |
| PR-7 | P7 | PAG-2 | Q1 | 145.57 | 6.32 |
| | (78.45) | (19.614) | (1.930) | | |
| PR-8 | P8 | PAG-2 | Q1 | 142.78 | 5.97 |
| | (78.45) | (19.614) | (1.930) | | |
| PR-9 | P9 | PAG-2 | Q1 | 161.50 | 6.17 |
| | (78.45) | (19.614) | (1.930) | | |

*denotes a comparative photoresist composition

The structure of the PAG (PAG-2) was as follows:

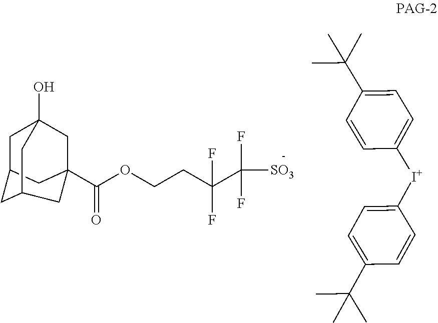

As shown above, the photoresist compositions PR-6 to PR-9 including a polymer derived from the inventive compound achieved improved LWR (i.e., a decreased LWR) and an improved E_{size} (increased E_{size}) relative to the photoresist composition that included the polymer that is not derived from the inventive compound.

### EUV Transmission Calculations

The effect of incorporating the inventive compounds on film absorption at EUV radiation is exemplified by the following transmission calculation results. The transmissions at EUV exposure (13.5 nm) for films made from composition examples PR-5 to PR-9 were calculated using the online calculation tool at the Center for X-Ray Optics at Lawrence Berkeley National Laboratory website by inputting the calculated composition molecular formula and assuming a film density of 1.30 g/cm³ and film thickness of 60 nm. The results are shown in Table 5 as percent transmittance (%).

TABLE 5

| Photoresist | Transmittance |
|---|---|
| Composition | (%) |
| PR-5* | 77.53 |
| PR-6 | 74.50 |
| PR-7 | 75.16 |
| PR-8 | 76.58 |
| PR-9 | 75.50 |

*denotes a comparative photoresist composition

As shown in Table 5, the inventive photoresist compositions PR-6 to PR-9 were calculated to have a greater absorption of 13.5 nm radiation as compared to PR-5.

1. A compound, comprising
an aromatic group or a heteroaromatic group, wherein the aromatic group or the heteroaromatic group comprises:
a first substituent group comprising an ethylenically unsaturated double bond,
a second substituent group that is an iodine atom, and
a third substituent group comprising an acid-labile group,
wherein the first sub stituent group, the second sub stituent group, and the third substituent group are each bonded to a different carbon atom of the aromatic group or the heteroaromatic group.

2. The compound of claim 1, wherein the first substituent group does not comprise an acid-labile group.

3. The compound of claim 1, wherein the compound is of Formula (1): 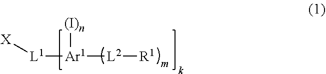
wherein, in Formula (1),
X is a polymerizable group comprising the ethylenically unsaturated double bond;
L¹ and L² are each independently a single bond or a divalent linking group;
Ar¹ is C₆₋₃₀ aryl or C₃₋₃₀ heteroaryl, each optionally further substituted with one or more of substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl;
R^{l} comprises the acid-labile group; and
n is an integer greater than or equal to 1;
m is an integer greater than or equal to 1;
provided n+m is an integer of 10 or less; and
k is an integer from 1 to 5.

4. The compound of claim 3, wherein R^{l} is represented by one of Formula (2) or (3): 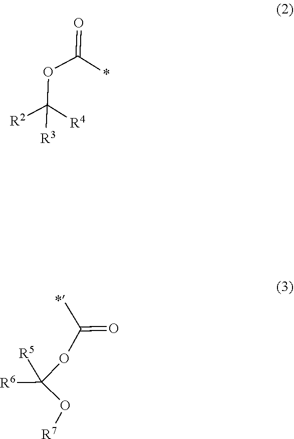
wherein, in Formulae (2) and (3),
R² to R⁴ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₂₋₂₀ heteroaryl, wherein each of R² to R⁴ optionally further comprises a divalent linking group as part of its structure;
provided that no more than one selected from R² to R⁴ is hydrogen, and provided that if one of R² to R⁴ is hydrogen, then at least one of the others from R² to R⁴ is substituted or unsubstituted C₆₋₂₀ aryl or substituted or unsubstituted C₃₋₂₀ heteroaryl;
any two of R² to R⁴ together optionally form a ring via a single bond or a divalent linking group, wherein the ring is substituted or unsubstituted;
R⁵ and R⁶ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein each of R⁵ and R⁶ optionally further comprises a divalent linking group as part of their structure;
R⁵ and R⁶ together optionally form a ring via a single bond or a divalent linking group, wherein the ring is substituted or unsubstituted;
R⁷ is substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein R⁷ optionally further comprises a divalent linking group as part of its structure;
any one or more of R⁵ or R⁶ together with R⁷ optionally form a ring via a single bond or a divalent linking group, wherein the ring is substituted or unsubstituted;
* and *′ each indicate a binding site to L².

5. The compound of claim 4, wherein
n is 1 or 2;
X is (meth)acrylic or substituted or unsubstituted C₂₋₁₂ alkenyl;
L¹ is a single bond;
L² is a single bond or —C(O))OC(X¹X²)—, wherein X¹ and X² are each independently hydrogen, fluorine, unsubstituted C₁₋₆alkyl, C1-6-fluoroalkyl, unsubstituted C₃₋₆ cycloalkyl, or C₃₋₆-fluorocycloalkyl;
Ar¹ is C₆₋₁₀ aryl, optionally further substituted with one or more of substituted or unsubstituted C₁₋₁₀ alkyl, substituted or unsubstituted C₁₋₁₀ heteroalkyl, substituted or unsubstituted C₃₋₁₀ cycloalkyl, or substituted or unsubstituted C₃₋₃₀ heterocycloalkyl;
R² to R⁴ are each independently hydrogen, substituted or unsubstituted C₁₋₁₀ alkyl, substituted or unsubstituted C³⁻⁸ cycloalkyl, or substituted or unsubstituted C₆₋₁₄ aryl;
provided that no more than one selected from R² to R⁴ is hydrogen, and provided that if one of R² to R⁴ is hydrogen, then at least one of the others from R² to R⁴ is substituted or unsubstituted C₆₋₁₄ aryl;
any two of R² to R⁴ together optionally form a ring via a single bond or a divalent linking group, wherein the ring is substituted or unsubstituted;
R⁵ and R⁶ are each independently hydrogen, or substituted or unsubstituted C₁₋₁₀ alkyl; and
R⁷ is substituted or unsubstituted C₁₋₁₀ alkyl, substituted or unsubstituted C³⁻⁸ cycloalkyl, or substituted or unsubstituted C₆₋₁₄ aryl.

6. A polymer, comprising a first repeating unit derived from the compound of claim 5.

7. The polymer of claim 6, wherein the polymer further comprises a second repeating unit comprising a tertiary alkyl ester group.

8. The polymer of claim 6, wherein the polymer further comprises a third repeating unit comprising a polar group pendant to a backbone of the polymer, and wherein the polar group is a lactone, hydroxy aryl, or fluoroalcohol group.

9. A photoresist composition, comprising:
the polymer of claim 6;
a photoacid generator; and
a solvent.

10. The photoresist composition of claim 9, further comprising a photo-decomposable quencher or a basic quencher.

11. A method for forming a pattern, the method comprising:
applying a layer of the photoresist composition of claim 9 on a substrate to provide a photoresist composition layer;
pattern-wise exposing the photoresist composition layer to activating radiation to provide an exposed photoresist composition layer; and
developing the exposed photoresist composition layer to provide a photoresist pattern.

12. The polymer of claim 6, wherein the first substituent group does not comprise an acid-labile group.

13. The polymer of claim 6, wherein the compound is of Formula (1): 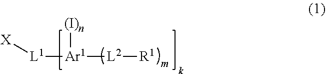
wherein, in Formula (1),
X is a polymerizable group comprising the ethylenically unsaturated double bond;
L¹ and L² are each independently a single bond or a divalent linking group;
Ar¹ is C₆₋₃₀ aryl or C₃₋₃₀ heteroaryl, each optionally further substituted with one or more of substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl;
R¹ comprises the acid-labile group; and
n is an integer greater than or equal to 1;
m is an integer greater than or equal to 1;
provided n+m is an integer of 10 or less; and
k is an integer from 1 to 5.

14. The polymer of claim 6, wherein le is represented by one of Formula (2) or (3): 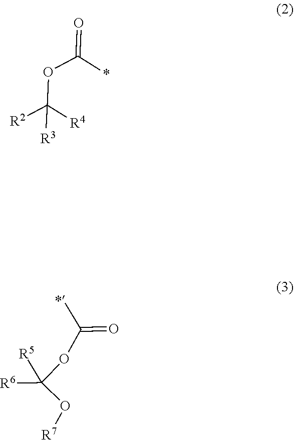
wherein, in Formulae (2) and (3),
R² to R⁴ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₂₋₂₀ heteroaryl, wherein each of R² to R⁴ optionally further comprises a divalent linking group as part of its structure;
provided that no more than one selected from R² to R⁴ is hydrogen, and provided that if one of R² to R⁴ is hydrogen, then at least one of the others from R² to R⁴ is substituted or unsubstituted C₆₋₂₀ aryl or substituted or unsubstituted C₃₋₂₀ heteroaryl;
any two of R² to R⁴ together optionally form a ring via a single bond or a divalent linking group, wherein the ring is substituted or unsubstituted;
R⁵ and R⁶ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein each of R⁵ and R⁶ optionally further comprises a divalent linking group as part of their structure;
R⁵ and R⁶ together optionally form a ring via a single bond or a divalent linking group, wherein the ring is substituted or unsubstituted;
R⁷ is substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein R⁷ optionally further comprises a divalent linking group as part of its structure;
any one or more of R⁵ or R⁶ together with R⁷ optionally form a ring via a single bond or a divalent linking group, wherein the ring is substituted or unsubstituted;
* and *′ each indicate a binding site to L².

15. The polymer of claim 6, wherein
n is 1 or 2;
X is (meth)acrylic or substituted or unsubstituted C₂₋₁₂ alkenyl;
L¹ is a single bond;
L² is a single bond or —C(O))OC(X¹X²)—, wherein X¹ and X² are each independently hydrogen, fluorine, unsubstituted C₁₋₆alkyl, C₁₋₆-fluoroalkyl, unsubstituted C₃₋₆ cycloalkyl, or C₃₋₆- fluorocycloalkyl;
Ar¹ is C₆₋₁₀ aryl, optionally further substituted with one or more of substituted or unsubstituted C₁₋₁₀ alkyl, substituted or unsubstituted C₁₋₁₀ heteroalkyl, substituted or unsubstituted C₃₋₁₀ cycloalkyl, or substituted or unsubstituted C₃₋₃₀ heterocycloalkyl;
R² to R⁴ are each independently hydrogen, substituted or unsubstituted C₁₋₁₀ alkyl, substituted or unsubstituted C³⁻⁸ cycloalkyl, or substituted or unsubstituted C₆₋₁₄ aryl;
provided that no more than one selected from R² to R⁴ is hydrogen, and provided that if one of R² to R⁴ is hydrogen, then at least one of the others from R² to R⁴ is substituted or unsubstituted C₆₋₁₄ aryl;
any two of R² to R⁴ together optionally form a ring via a single bond or a divalent linking group, wherein the ring is substituted or unsubstituted;
R⁵ and R⁶ are each independently hydrogen, or substituted or unsubstituted C₁₋₁₀ alkyl; and
R⁷ is substituted or unsubstituted C₁₋₁₀ alkyl, substituted or unsubstituted C³⁻⁸ cycloalkyl, or substituted or unsubstituted C₆₋₁₄ aryl.

16. The photoresist composition of claim 9, wherein the first substituent group does not comprise an acid-labile group.

17. The photoresist composition of claim 9, wherein the compound is of Formula (1): 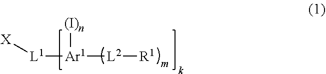
wherein, in Formula (1),
X is a polymerizable group comprising the ethylenically unsaturated double bond;
L^{l} and L² are each independently a single bond or a divalent linking group;
Ar¹ is C₆₋₃₀ aryl or C₃₋₃₀ heteroaryl, each optionally further substituted with one or more of substituted or unsubstituted C₁₋₃₀ alkyl, substituted or unsubstituted C₁₋₃₀ heteroalkyl, substituted or unsubstituted C₃₋₃₀ cycloalkyl, substituted or unsubstituted C₁₋₃₀ heterocycloalkyl, substituted or unsubstituted C₂₋₃₀ alkenyl, substituted or unsubstituted C₂₋₃₀ alkynyl, substituted or unsubstituted C₆₋₃₀ aryl, substituted or unsubstituted C₇₋₃₀ arylalkyl, substituted or unsubstituted C₇₋₃₀ alkylaryl, substituted or unsubstituted C₃₋₃₀ heteroaryl, substituted or unsubstituted C₄₋₃₀ alkylheteroaryl, or substituted or unsubstituted C₄₋₃₀ heteroarylalkyl;
R¹ comprises the acid-labile group; and
n is an integer greater than or equal to 1;
m is an integer greater than or equal to 1;
provided n+m is an integer of 10 or less; and
k is an integer from 1 to 5.

18. The photoresist composition of claim 9, wherein le is represented by one of Formula (2) or (3): 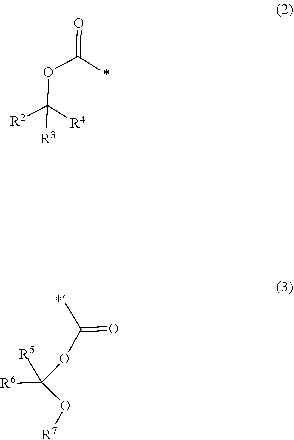
wherein, in Formulae (2) and (3),
R² to R⁴ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₂₋₂₀ alkenyl, substituted or unsubstituted C₃₋₂₀ cycloalkenyl, substituted or unsubstituted C₃₋₂₀ heterocycloalkenyl, substituted or unsubstituted C₆₋₂₀ aryl, or substituted or unsubstituted C₂₋₂₀ heteroaryl, wherein each of R² to R⁴ optionally further comprises a divalent linking group as part of its structure;
provided that no more than one selected from R² to R⁴ is hydrogen, and provided that if one of R² to R⁴ is hydrogen, then at least one of the others from R² to R⁴ is substituted or unsubstituted C₆₋₂₀ aryl or substituted or unsubstituted C₃₋₂₀ heteroaryl;
any two of R² to R⁴ together optionally form a ring via a single bond or a divalent linking group, wherein the ring is substituted or unsubstituted;
R⁵ and R⁶ are each independently hydrogen, substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein each of R⁵ and R⁶ optionally further comprises a divalent linking group as part of their structure;
R⁵ and R⁶ together optionally form a ring via a single bond or a divalent linking group, wherein the ring is substituted or unsubstituted;
R⁷ is substituted or unsubstituted C₁₋₂₀ alkyl, substituted or unsubstituted C₃₋₂₀ cycloalkyl, substituted or unsubstituted C₃₋₂₀ heterocycloalkyl, substituted or unsubstituted C₆₋₂₀ aryl, or substituted or unsubstituted C₂₋₂₀ heteroaryl, wherein R⁷ optionally further comprises a divalent linking group as part of its structure;
any one or more of R⁵ or R⁶ together with R⁷ optionally form a ring via a single bond or a divalent linking group, wherein the ring is substituted or unsubstituted;
* and *′ each indicate a binding site to L².

19. The photoresist composition of claim 9, wherein
n is 1 or 2;
X is (meth)acrylic or substituted or unsubstituted C₂₋₁₂ alkenyl;
L¹ is a single bond;
L² is a single bond or —C(O))OC(X′X²)—, wherein X¹ and X² are each independently hydrogen, fluorine, unsubstituted C₁₋₆alkyl, C₁₋₆-fluoroalkyl, unsubstituted C₃₋₆ cycloalkyl, or C₃₋₆- fluorocycloalkyl;
Ar¹ is C₆₋₁₀ aryl, optionally further substituted with one or more of substituted or unsubstituted C₁₋₁₀ alkyl, substituted or unsubstituted C₁₋₁₀ heteroalkyl, substituted or unsubstituted C₃₋₁₀ cycloalkyl, or substituted or unsubstituted C₃₋₃₀ heterocycloalkyl;
R² to R⁴ are each independently hydrogen, substituted or unsubstituted C₁₋₁₀ alkyl, substituted or unsubstituted C³⁻⁸ cycloalkyl, or substituted or unsubstituted C₆₋₁₄ aryl;
provided that no more than one selected from R² to R⁴ is hydrogen, and provided that if one of R² to R⁴ is hydrogen, then at least one of the others from R² to R⁴ is substituted or unsubstituted C₆₋₁₄ aryl;
any two of R² to R⁴ together optionally form a ring via a single bond or a divalent linking group, wherein the ring is substituted or unsubstituted;
R⁵ and R⁶ are each independently hydrogen, or substituted or unsubstituted C₁₋₁₀ alkyl; and
R⁷ is substituted or unsubstituted C₁₋₁₀ alkyl, substituted or unsubstituted C³⁻⁸ cycloalkyl, or substituted or unsubstituted C₆₋₁₄ aryl.
